(19) United States
(12) Patent Application Publication
(10) Pub. No.: US 2026/0363600 A1
(43) Pub. Date: Nov. 19, 2026

(54) SYSTEMS AND METHODS FOR BRAIN STATE STIMULATION

(71) Applicant: Duke University, Durham, NC (US)

(72) Inventors: Kafui Dzirasa, Durham, NC (US); David Carlson, Durham, NC (US); Austin Talbot, Durham, NC (US); Neil Gallagher, Durham, NC (US); Yael Grossman, Durham, NC (US)

(21) Appl. No.: 19/136,545

(22) PCT Filed: Dec. 7, 2023
(86) PCT No.: PCT/US2023/082965

§ 371 Date: Jun. 6, 2025

Related U.S. Application Data
(60) Provisional application No. 63/430,691, filed on Dec. 7, 2022.

(51) Int. Cl. A61B 5/369; A61N 1/05; A61N 1/36

(52) U.S. Cl. CPC A61B 5/369; A61N 1/0529; A61N 1/36078; A61N 1/36082; A61N 1/36139; A61N 1/36167

(57) ABSTRACT

The present disclosure provides systems and methods for predicting and treating a neurological behavior in a subject. The system may include a plurality of electrodes configured to receive electrical activity signals from a network of two or more regions of the subject's brain and a controller in communication with the plurality of electrodes. The system may be configured to receive and record the electrical activity signals from the plurality of electrodes, provide the electrical activity signals to the controller, generate a prediction of a time of onset of the neurological behavior, and send a stimulation signal to the brain prior to the predicted time of onset of the neurological behavior. The stimulation signal to the brain may suppress the neurological behavior.

### CROSS-REFERENCE TO RELATED APPLICATIONS

This application claims the benefit of and incorporates by reference the content of U.S. Provisional App. No. 63/430,691, filed Dec. 7, 2022.

### GOVERNMENT LICENSE RIGHTS

This invention was made with government support under Federal Grant Nos. R01MH120158 and 1R01MH125430 awarded by the National Institute of Mental Health (NIH/NIMH) and Federal Grant no. 1R01EB026937 awarded by the National Institute of Biomedical Imaging and Bioengineering (NIBIB). The government has certain rights to this invention.

### BACKGROUND

Social behavior reflects the integration of sensory information with internal affective states. Many subcortical brain regions contribute to aggressive behavior in mammals including lateral septum (LSN), nucleus accumbens, lateral habenula, the ventrolateral portion of ventromedial hypothalamus, and medial amygdala. Prefrontal cortex stimulation has been shown to mitigate aggressive behavior in both humans, implicating cortical regions in regulating aggression. Finally, sensory regions, such as those underlying olfaction, also contribute to aggression.

To appropriately regulate aggressive behavior, the brain must integrate information across these and other cortical and subcortical regions. Moreover, since many of these regions also regulate non-aggressive social behaviors, the brain must ultimately utilize overlapping sources of information to segregate aggression from other social behavioral states. While this points to the existence of a broader brain-network level process underlying aggression, such a mechanism that generalizes across animals remains unknown. Addressing this question is of major importance as 1) mammals regularly select from a repertoire of social behavioral states based on external sensory cues to ensure their survival, and 2) a range of psychiatric disorders are broadly marked by a failure to appropriately match behavior with evolving social contexts.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The present disclosure provides a system for predicting and treating a neurological behavior in a subject. The system may include a plurality of electrodes configured to receive electrical activity signals from a network of two or more regions of the subject's brain; and a controller in communication with the plurality of electrodes, the controller having at least one processor in communication with a memory, the memory including instructions executable by the processor to: receive and record the electrical activity signals from the plurality of electrodes; generate a prediction of a time of onset of the neurological behavior from the electrical activity signals; and send a stimulation signal to the brain prior to the predicted time of onset of the neurological behavior. The stimulation signal to the brain suppresses the neurological behavior.

In an aspect, the memory may include further instructions to: provide the electrical activity signals to a trained machine learning model, wherein the prediction of the time of onset is generated from the trained machine learning model.

In some aspects of the present disclosure, the plurality of electrodes are microwire electrodes. The plurality of electrodes may be implanted in the subject's brain.

In some aspects, the two or more regions comprises 8 to 11 regions of the subject's brain. The two or more brain regions may be selected from infralimbic cortex, orbitofrontal cortex, prelimbic cortex, lateral septum, nucleus accumbens, lateral habenula, mediodorsal thalamus, ventromedial hypothalamus, medial amygdala, primary visual cortex, ventral hippocampus, and combinations thereof. In some aspects, the neurological behavior is an aggressive state or an aggressive attack.

In some aspects of the present disclosure, the time of onset of the neurological behavior is on a second timescale. The system may further include a stimulator configured to generate the stimulation signal. In an aspect, the stimulation signal may be light stimulation. In an example, the stimulation signal may include closed loop optogenetic stimulation. The stimulation signal may be directed to the prefrontal cortex of the subject's brain.

Further provided herein is a method for predicting and treating a neurological behavior in a subject including: providing a plurality of electrodes configured to receive electrical activity signals from a network of two or more regions of the subject's brain; receiving and recording the electrical activity signals from the plurality of electrodes, via a controller in communication with the plurality of electrodes; generating a prediction of a time of onset of the neurological behavior from the electrical activity signals; and sending a stimulation signal to the brain prior to the predicted time of onset of the neurological behavior, where the stimulation signal to the brain suppresses or prevents onset of the neurological behavior.

The method may further include providing the electrical activity signals to a trained machine learning model, wherein the prediction of the time of onset is generated from the trained machine learning model.

In some aspects, the two or more regions may include 8 to 11 regions of the subject's brain. The two or more brain regions may be selected from infralimbic cortex, orbitofrontal cortex, prelimbic cortex, lateral septum, nucleus accumbens, lateral habenula, mediodorsal thalamus, ventromedial hypothalamus, medial amygdala, primary visual cortex, and ventral hippocampus. In some aspects, the neurological behavior is an aggressive state or an aggressive attack.

In an aspect, the plurality of electrodes may be implanted in the subject's brain. The time of onset of the neurological behavior may be on a second timescale. In some aspects, the stimulation signal is directed to the prefrontal cortex of the subject's brain. In additional aspects, the stimulation signal may include closed loop optogenetic stimulation.

These as well as other embodiments, aspects, advantages, and alternatives will become apparent to those of ordinary skill in the art by reading the following detailed description, with reference where appropriate to the accompanying drawings. Further, it should be understood that this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF THE FIGURES

FIG. 1 illustrates a schematic diagram of a system, according to an example embodiment.

FIG. 2 illustrates example system embodiments.

FIG. 3 illustrates an example machine learning environment.

FIG. 4 is an example method in one embodiment.

FIG. 5A is a schematic of optogenetic stimulation and shows social encounters utilized for testing.

FIG. 5B shows prefrontal cortex stimulation suppressed attack behavior, increased non-attack social behavior towards male mice, and suppressed non-attack social behavior towards females (*P<0.05 for each comparison).

FIG. 5C shows example targeted brain regions.

FIG. 5D shows representative local field potentials recorded during repeated exposure to social contexts that produce attack and non-attack social behavior.

FIG. 5E shows a framework to test encoding of social states by individual brain regions.

FIG. 5F shows all implanted regions encoded social engagement; however, only five selectively encoded the attack behavior. Pink shading indicates P<0.05 with FDR correction.

FIG. 5G shows attack codes discovered from the five brain regions failed to encode aggressive behavior induced by male urine (Gray shading indicates P<0.05 prior to but not following FDR correction).

FIG. 5H is a schematic of a machine-learning model used to discover network encoding attack behavior and encoding across eight learned networks.

FIG. 5I shows the supervised network (purple, EN-AggINH) showed the strongest encoding. Data shown as mean±SEM.

FIG. 6A shows neural activity was sampled while mice were socially isolated and during intervals surrounding social behavior.

FIG. 6B shows network activity during this interval encoded attack behavior vs. male and female non-attack social behaviors, while ventral hippocampal activity did not (P<0.05 using Freidman's test followed by Wilcoxon sign-rank test). Network during isolation, but not ventral hippocampus activity, encoded the subsequent total attack time of individual mice (P<0.05 using Spearman's Rank Correlation).

FIG. 7A shows prominent oscillatory frequency bands composing EN-AggINH are highlighted for each brain region around the rim of the circle plot. Prominent synchrony measures are depicted by lines connecting brain regions through the center of the circle. The plot is shown at relative spectral energy of 0.4. Theta (4-11 Hz) and beta (14-30 Hz) frequency components are highlighted in blue and green, respectively.

FIG. 7B shows example relative LFP spectral energy plots for three brain regions corresponding to the circular plot in FIG. 7A.

FIG. 7C shows Granger offset measures were used to quantify directionality within EN-AggINH. Prominent directionality was observed across the theta and beta frequency bands (shown at spectral density threshold of 0.4 and a directionality offset of 0.3).

FIG. 7D is a schematic depicting directionality within EN-AggINH.

FIG. 7E a representative cell showing firing activity that is negatively correlated with EN-AggINH activity.

FIG. 7F shows EN-AggINH activity correlated with cellular firing across the brain across the brain.

FIG. 8A shows an example strategy for validating EN-AggINH using additional aggression paradigms.

FIG. 8B shows an experimental approach for causally inducing aggression via direct cellular manipulation.

FIG. 8C shows cellular activation induced attack behavior towards female mice (P<0.001 using sign-rank test).

FIG. 8D shows decreased EN-AggINH activity across during social interactions with female mice (P<0.01 using one-tailed Wilcoxon sign-rank test).

FIG. 8E shows EN-AggINH activity and intervals surrounding these interactions (P<0.05 using Wilcoxon sign-rank test).

FIG. 9A is a schematic for closed loop manipulation of EN-AggINH activity.

FIG. 9B is an example real-time estimation of aggression. Receiver operating characteristic depicting detection of aggressive behavior in a mouse using EN-AggINH activity vs. real-time reduced encoder is shown to the right. Dashed blue line corresponds to the established detection threshold.

FIG. 9C shows detection of aggression using reduced encoder vs. EN-AggINH across mice (N=9; P=0.43 using two-tailed paired Wilcoxon sign-rank).

FIG. 9D shows EN-AggINH activity relative to light stimulation during closed loop manipulation.

FIG. 9E shows a portion of windows stimulated during social behaviors (P=0.002 using one-tailed sign-rank test).

FIG. 9F shows behavioral effects of closed loop stimulation (** P<0.005 using paired t-test, significance determined using FDR correction).

FIG. 9G is a directed graph with the inferred modes of action derived from mediation analysis. There is a causal relationship from stimulation to behavior and stimulation to EN-AggINH expression (model 1; P<0.01 using signed rank and paired t-tests). EN-AggINH is a mediator from stimulation to behavior (P<0.01 using nested logistic regression models, likelihood ratio test; model 2), EN-AggINH activation is the primary mechanism whereby prefrontal cortex stimulation suppresses aggression (P<0.01 using average causal mediation effect, model 3).

FIG. 9H is a schematic for nonsynchronous control stimulation.

FIG. 9I shows nonsynchronous stimulation does not impact aggressive behavior (P>0.05 using paired t-test).

FIG. 9J shows an impact of open loop (20 Hz, fixed) and closed loop stimulation on locomotor behavior (P**<0.01 using paired two-tailed t-test).

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features. Other embodiments can be utilized, and other changes can be made, without departing from the scope of the subject matter presented herein.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. As used herein, the terms “prevent,” “preventing,” “prevention,” “prophylactic treatment” and the like refer to reducing the probability of developing a disease, disorder or condition in a subject, who does not have, but is at risk of or susceptible to developing a disease, disorder or condition. The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient).

Unless otherwise defined, all technical terms used herein have the same meaning as commonly understood by one of ordinary skill in the art to which this disclosure belongs.

Thus, the example embodiments described herein are not meant to be limiting. Aspects of the present disclosure, as generally described herein, and illustrated in the figures, can be arranged, substituted, combined, separated, and designed in a wide variety of different configurations, all of which are contemplated herein.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

### Overview

Since many brain regions contribute to multiple social behaviors, expanded knowledge of how the brain encodes distinct social states would enable the development of interventions that suppress aggression, while leaving other social behaviors intact. Hence, the present disclosure is based, in part, on the finding by the inventors that murine aggression is optimally encoded by a brain-wide network and that this network is organized by prominent theta (4-11 Hz) and beta (14-30 Hz) oscillations, leading from orbital frontal cortex and medial dorsal thalamus, and converging on ventral medial hypothalamus and medial amygdala. Activity in this network was shown to be coupled to brain-wide cellular firing, and the network is conserved in new mice from multiple genetic backgrounds, and in multiple contexts. In some examples, the network may be a network that encodes aggression (e.g. EN-AggINH).

Accordingly, one aspect of the present disclosure provides a method of reducing and/or eliminating aggression in a subject, the method comprising, consisting of, or consisting essentially of increasing activity in a network of brain regions (e.g. EN-AggINH activity) in the brain of the subject such that the aggression in the subject is suppressed, reduced and/or eliminated.

In some embodiments, the EN-AggINH activity is increased by applying one or more light stimulations. In one embodiment, the light stimulation includes 5 mW, 20 Hz, 3 ms pulse width. In another embodiment, the stimulation includes a 20 Hz fixed frequency stimulation of infralimbic cortex.

Another aspect of the present disclosure provides for a closed-loop stimulation protocol based on network activity levels that suppresses aggression, but not pro-social behavior, in a subject, the method comprising, consisting of, or consisting essentially of applying to the subject one or more closed loop stimulations of the infralimbic cortex when EN-AggINH activity decreases below a threshold level as compared to a control.

In some embodiments, the closed-loop stimulation includes one or more light stimulations. In one embodiment, the light stimulation includes 5 mW, 20 Hz, 3 ms pulse width. In another embodiment, the stimulation includes a 20 Hz fixed frequency stimulation of infralimbic cortex. Another aspect of the present disclosure provides a designer receptor exclusively activated by designer drug (DREADD) based approach to selectively activate cells in ventral medial hypothalamus in a manner that has been shown to induce aggression.

Treatment of such neurological behavior, psychological disorders or conditions may include a variety of interventions to reverse such changes and/or compensate for the effects of such changes. Treatments may include talk therapy, pharmaceutical intervention, surgical intervention, light stimulation, electrical stimulation, or some other procedures or therapies. Treatments may act to compensate for changes underlying a neurological behavior or condition (e.g., by increasing the availability of a neurotransmitter by reducing the rate of reuptake of the neurotransmitter, by increasing the intrinsic activity of a brain region, by blocking the activity of a brain region and/or blocking efferents from the brain region) and/or to reverse the changes underlying the neurological behavior, psychological disorder or condition.

Stimulating a brain region, based on the detected electrical activity signals from a network of two or more regions of the brain, could involve providing electrical signals (e.g., pulses or other waveforms of current and/or voltage), optical stimulation (e.g., to neurons that are sensitive to optical stimulation and/or that have been made sensitive by some intervention), chemical stimulation (e.g., by emission of timed amounts of a stimulating neurotransmitter or other substance), or providing some other excitatory stimulus in a manner that is timed according to the predicted onset time of the behavior or other information related to detected activity of the network. Provided stimuli could include square pulses, triangular pulses, or some other waveforms or patterns of stimulation.

Systems and devices configured to affect the therapies and methods described herein (e.g., to detect timing information about the onset of a neurological behavior and to provide excitatory stimuli to a brain region based on the detected timing information) could include implanted and/or non-implanted elements. Implanted elements could include electrodes, optical fibers, leads extending beneath the skin between elements of the device or system, leads or connectors traversing the skin to permit connections between implanted and non-implanted elements, controllers, batteries, communications or power transfer coils, or other elements. Non-implanted elements could include wearable electrodes or devices (e.g., an array of electroencephalogram (EEG) electrodes configured to be worn as a cap), coils for the delivery of transcranial magnetic stimulation (TMS), magnetic resonance imagers (MRI), magnetoencephalography (MEG) equipment, control interfaces (e.g., for use in setting parameters of operation of the system by a clinician or other user), power sources, or other elements. Implanted and non-implanted elements may be in wireless or wired communication (e.g., an implanted device that is configured to detect activity in a first brain region could communicate phase or other timing information about the detected activity to a TMS stimulator that is located outside of the body) and/or may be in communication with other systems (e.g., a computer in a physician's office).

The devices, systems, and methods described herein are not limited to the detection of activity in a single brain region and providing excitatory stimuli, based on the detected activity, to a single further brain region. Stimuli could be provided to one or more brain regions based on the timing or other properties of activity detected in one or more brain regions. Stimuli could be provided and/or activity detected in a brain region on one side of the brain (unilaterally) or on both sides of the brain (bilaterally). Other patterns of detection from one or more regions and providing stimuli to one or more further regions and/or to the regions from which the activity is detected are anticipated by the inventors.

### II. Systems

A variety of devices and systems can be used for predicting and treating a neurological behavior in a subject. Provided herein is an exemplary system for predicting and treating a neurological behavior in a subject. The system can include a plurality of electrodes configured to receive electrical activity signals from a network of two or more regions of the subject's brain and a controller in communication with the plurality of electrodes. The neurological behavior may be any neurological behavior detectable from the plurality of electrodes within the network of brain regions. Non-limiting examples of neurological behaviors include an aggressive state, an aggressive attack, appetitive social behavior, and depressive behavior.

Such systems can include elements that are implanted within a body (e.g., electrodes implanted within a brain) and/or elements that are disposed outside of the body (e.g., electrodes or other elements that may be removably mounted to the skin of the body). Elements that are implanted within a body could be operably coupled to elements outside of the body (e.g., mounted to a surface of the body) by cabling, connectors, or other means passing through the skin. Additionally or alternatively, elements of such systems could be wirelessly coupled, e.g., by coils, antenna, or other means for transmitting and/or receiving wireless communications and/or wireless power. Such systems could be configured for chronic use (e.g., to provide stimulation over a protracted period of time) or for acute and/or periodic use.

Such systems can be configured to detect a variety of different signals in a network of two or more brain regions. For example, such systems can be configured to detect electrical current or voltages, electrical fields, magnetic fields, chemical concentrations or gradients, acoustical waves, temperatures or temperature gradients, the intensity or wavelength of light emitted from a brain region (e.g., in response to illumination or some other applied energy or generated internally by the brain region), or some other physical variable related to the activity of a brain region. The electrical signals from the network of two or more regions of the brain may provide more information for predicting an onset of the neurological behavior than electrical signals from one region alone. In an example, the network can include 2 or more, 3 or more, 4 or more, 5 or more, 6 or more, 7 or more, 8 or more, 9 or more, 10 or more, or 11 or more regions of the subject's brain. In other examples, the network can include up to 2, up to 3, up to 4, up to 5, up to 6, up to 7, up to 8, up to 9, up to 10, or up to 11 regions of the subject's brain. In at least one example, the network can include 8 to 11 regions of the subject's brain. The two or more regions of the brain in the network may include but are not limited to infralimbic cortex, orbitofrontal cortex, prelimbic cortex, lateral septum, nucleus accumbens, lateral habenula, mediodorsal thalamus, ventromedial hypothalamus, medial amygdala, primary visual cortex, and ventral hippocampus. In at least one example, the network may be a network that encodes aggression (e.g. an electome network referred to as EN-Aggression Inhibition-AggINH; “EN-AggINH”).

Furthermore, such systems can be configured to provide a variety of different types of stimuli (e.g. stimulation signal) to excite a brain region. For example, such systems can be configured to apply electrical currents or voltages, time-varying magnetic fields, radio frequency waves or fields, light (e.g., infrared light, ultraviolet light, or visible light to stimulate an optically-sensitive substance present in a brain region), chemical substances (e.g., neurotransmitters), acoustical waves, or other energies or substances that can excite activity in elements (e.g., cells, axons) of the brain. In some examples, such systems can include a stimulator for applying the stimulation signal.

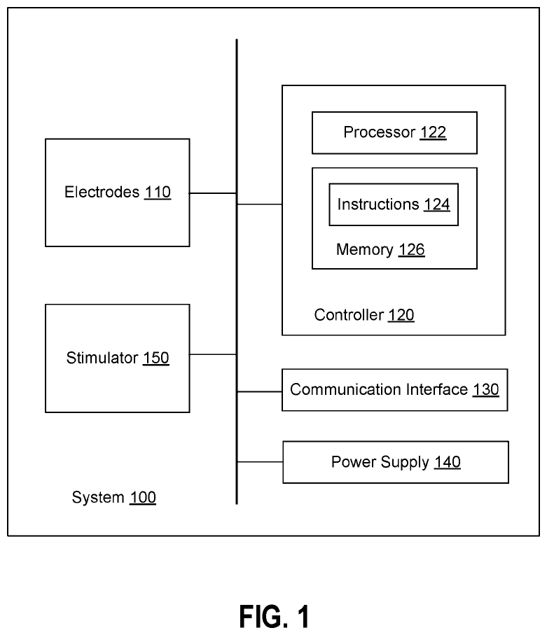

FIG. 1 illustrates a schematic diagram of a system 100, according to an example embodiment. System 100 includes a plurality of electrodes 110 and a controller 120. System 100 can also include a stimulator 150, a communication interface 130, and a power supply 140. The plurality of electrodes 110 are configured to detect electrical activity signals from a network of two or more regions of the brain (e.g., to detect a local field potential, an electroencephalogram, an electrical field generated by the electrical activity of one or more neurons, or some other electrical voltage or current related to the electrical activity of the brain region). In some examples, the plurality of electrodes can be microwire electrodes. The electrical activity signals can include prominent theta (4-11 Hz) and beta (14-30 Hz) oscillations from the network of two or more regions of the brain, but signals that include components at other frequencies are possible.

It is understood that some or all elements of system 100 may be implantable in a patient. Furthermore, at least some elements of system 100 may remain external to, and/or may be worn by, the patient. Further, individual illustrated components (e.g., the plurality of electrodes 110, the stimulator 150, the controller 120) may be distributed and/or duplicated across multiple elements of the system 100. For example, the plurality of electrodes 110, the stimulator 150, and/or elements of the controller 120 may be disposed in an implanted device while the stimulator 150 and/or other elements of the controller 120 may be disposed in an external device that is configured to be mounted to or otherwise maintained in position relative to the brain of a person, outside of the body of the person. In such an example, the implanted device can be configured to detect electrical activity signals from a network of two or more regions of the brain (e.g., by transmitting the detected signals) to the external device. The external device could then operate to provide excitatory stimulus (e.g. stimulation signal) to a brain region, in the form of optogenetic stimulation, based on the received electrical activity signals and predicted time of onset of the neurological behavior (e.g., such that the excitatory stimulus is provided at a set time or range of times prior to the predicted time of onset).

The plurality of electrodes 110 could be configured to detect such signals from outside of the brain. This could include detecting an electrical and/or magnetic field outside of the skull (e.g., using a magnetoencephalogram), detecting an electrical field and/or current on or within the scalp (e.g., using one or more electrodes disposed on or within the scalp to detect an electroencephalogram), detecting an electrical field and/or current at one or more locations within or beneath the skull (e.g., using one or more electrodes disposed on the dura and/or beneath the dura on the surface of the brain or within the scalp to detect an electroencephalogram and/or a local field potential), or detecting some other signal of interest outside of the brain. Additionally or alternatively, the plurality of electrodes (or sensors) 110 could be configured to detect signals within the brain. This could include detecting an electrical field and/or current within the cortex of the brain (e.g., using two or more penetrating electrodes), detecting light emitted and/or reflected from within the cortex of the brain (e.g., using a penetrating device that includes an optical fiber or other means for detecting such a light), detecting an electrical field and/or current within a deep structure of the brain (e.g., using one or more electrodes disposed on a lead penetrating from the surface of the brain to the deep structure), detecting light emitted and/or reflected from a deep structure of the brain (e.g., using a device that penetrates from the surface of the brain to the deep structure and that includes an optical fiber or other means for detecting such a light), or detecting some other signal(s) from within the brain.

The controller 120 is configured to receive and record the electrical activity signals from the plurality of electrodes 110. In some examples, the controller 120 can determine when there is a change in the electrical activity signals of the network that indicates a behavior change. For example, a reduction in the electrical signal from the network can indicate an onset of a neurological behavior, such as an aggressive state. In some examples, the reduction can be determined by comparing the signals to a threshold. Once the signals are below the threshold, the controller may predict a time of onset of the neurological behavior. The threshold can be determined from a baseline of electrical activity signals from the network in the absence of the neurological behavior. A predicted time of onset of the neurological behavior may be determined from the reduction in the network electrical signal. In additional examples, the electrical activity signals can be provided to a trained machine learning model that generates a prediction of a time of onset of the neurological behavior from the trained machine learning model. In various examples, the predicted time of onset of the neurological behavior is on a second timescale. For example, the predicted time of onset may be provided about 1 s, about 2 s, about 3 s, about 4 s, about 5 s, about 6 s, about 7 s, about 8 s, about 9 s, or about 10 s before the neurological behavior manifests.

In some examples, the system 100 can further include a stimulator 150. The controller can further be configured to send a stimulation signal (e.g. light stimulation, electrical stimulation, etc.) to the brain prior to the predicted time of onset of the neurological behavior. The stimulator 150 can be configured to generate the stimulation signal. The stimulation signal to the brain suppresses the neurological behavior. The stimulator 150 is configured to provide an excitatory stimulus (e.g. stimulation signal) to a brain region (e.g., an electrical voltage and/or current, an optical/light signal, channel, or other element(s) of the desired brain region). The brain region that the stimulation signal is applied to can be a same or different brain region with two or more electrodes. The stimulation signal can be directed to the prefrontal cortex of the subject's brain. In an example, the stimulation signal includes closed loop optogenetic stimulation. In other examples, the stimulator 150 is one or more of the plurality of electrodes 110. In other examples, the stimulator 150 provides optogenetic stimulation.

The system 100 can further include leads, optical fibers, or other means for transmitting a signal of interest from a brain region that is generating the signal to the controller 120. These means of transmitting a signal may be integral with the plurality of electrodes 110. In some examples, the controller can include a data acquisition system 120. Additionally or alternatively, elements of the data acquisition system or controller 120 may be disposed proximate to the brain region generating such a signal. For example, amplifiers, filters, analog-to-digital converters, photodetectors, light emitters, integrated circuits, or other electronic elements may be disposed on the backside of one or more surface electrodes, proximate the tip of a device configured to penetrate from the surface of the brain to a deep structure of the brain, or at some other location proximate a brain region of interest and/or elements of the electrodes 110 (e.g., electrodes, optical fibers, lenses, chemical transducers) configured to detect an electrical activity signal from the brain regions.

The plurality of electrodes 110 may include two or more electrodes configured to conduct electrical current and/or measure a local field potential (LFP) within two or more brain regions. An LFP may be a signal generated by one or more neurons proximate to an electrode 110. In some embodiments, the LFP signal may provide information about physiology such as: synaptic currents, neuronal connectivity, average neural activity, a degree of coherence and/or synchronization of activity of a population of neurons, and/or neural interaction within a given brain region. The electrical activity signals may include oscillating signals having frequencies between 3-30 Hz, 3-6 Hz, 4-11 Hz, and/or 14-30 Hz, but signals that include components at other frequencies are possible.

In some embodiments, at least a portion of the plurality of electrodes 110 may be implanted via a burr hole in a patient's skull. Upon proper delivery/placement of the electrodes 110, a portion of the electrodes 110 may be anchored to the patient's skull. The plurality of electrodes 110 may be communicatively coupled to the controller 120 via a wireless or wired connection, which may include communication interface 130.

The controller 120 is configured to detect, using the electrodes 110, electrical activity signals that may be related to the activity of two or more brain regions within a predetermined network of brain regions. The controller 120 can include a data acquisition system that includes a single or multi-channel recording system; that is, the data acquisition system may be configured to detect electrical activity signals (or other signals relating to such oscillatory signals) from one or more electrodes and/or elements of the electrodes (e.g., one or more electrodes of an electrode array, one or more electrodes of a tetrode). In an example embodiment, the controller 120 or data acquisition system may receive electrical signals from the plurality of electrodes 110. In an example, the controller 120 or data acquisition system may convert an analog voltage waveform to a digital signal. For example, the controller 120 or data acquisition system may include one or more voltmeters or analog-to-digital converter circuits, which may provide information indicative of one or more stimulation signals in units of volts. In some examples, the controller 120 may be configured to operate one or more light emitters, piezoelectric transducers, electrodes, or other elements configured to emit an energy into the brain region (e.g., light energy, acoustical energy) to increase the activity within the network to suppress the neurological behavior.

The controller 120 may include a digital signal processing system. In some embodiments, the controller 120 may provide one or more clock and/or trigger signals to another element of system 100, such as the stimulator 150. The controller 120 may be configured to record and store signals locally and/or in the memory 154.

The stimulator 150 may be configured to provide electrical, optical, chemical, acoustical, or some other variety of excitatory stimuli (e.g. stimulation signal) to a brain region according to one or more outputs of the controller 120. The stimulator 150 can include light emitters, electrodes, piezoelectric transducers, chemical transducers, or other tissue-stimulating elements. The stimulator 150 can include amplifiers, filters, transducers, digital-to-analog converters, buffers, or other elements configured to convert, condition, or otherwise modify signals sent or generated by the controller 120 such that the stimulator 150 provides at least one stimulus to a brain region. The stimulator 150 can include blocking capacitors, resistors, fuses, clamping diodes, or other elements to prevent the application of deleterious stimuli to the desired brain region (e.g., to limit a maximum current and/or voltage applied, by electrodes of the stimulator 150, to the brain). The stimulator 150 can include wires, optical fibers, lenses, leads, waveguides, or other elements configured to transmit a generated electrical voltage or current, a light, an acoustical energy, or some other stimulus signal to the brain region from an emitting element of the system 100 (e.g., from a light emitter, electrical buffer or amplifier, or other element(s) of the stimulator 150).

In an example embodiment, the brain region receiving the stimulation signal may be the same or different from the two or more brain regions in the network from which the electrical activity signals are obtained. In such a scenario, stimulator 150 may include one or more optical fibers, wires connected to one or more electrodes, or other elements that may be proximate to the desired brain region for stimulation. The stimulator 150 may provide voltage waveforms, light intensity waveforms, of other signals to the brain region. In some embodiments, stimulator 150 may include circuitry such as filters, amplifiers, etc.

Stimulator 150 may be similar or identical to at least one of the plurality of electrodes 110. For example, stimulator 150 and electrodes 110 could both include respective one or more electrodes configured to provide an electrical interface with tissues of the brain. Furthermore, stimulator 150 may be incorporated into at least some elements of at least one electrode of the plurality of electrodes 110. In an example embodiment, stimulator 150 and at least one electrode of the plurality of electrodes 110 may be combined into a single implantable device. Furthermore, by time division, frequency division, or another controllable method, one or more of the electrodes of the implantable device may act as either a sensing electrode or a stimulating electrode. For example, an electrode of the stimulator 150 can be used both to provide an excitatory electrical stimulus to a desired brain region and to detect an electrical signal from one or more brain regions in a network. For example, a detected electrical activity signal can be used to determine a time of onset of a behavior and to control stimulation signal provided to the desired brain region using the stimulator 150. In other embodiments, stimulator 150 may take the form of an implantable device that is separate with respect to the plurality of electrodes 110.

In some examples, the stimulation signal is light stimulation. The stimulation signal may be directed to the prefrontal cortex of the subject's brain. In an example, the stimulation signal includes closed loop optogenetic stimulation. The amount of closed loop optogenetic light stimulation may be varied depending on the particular need of the subject. In some examples, the closed loop stimulation includes about 1 second, about 2 seconds, about 3 seconds, about 4 seconds, or about 5 seconds of light stimulation. In at least one example, the closed loop stimulation includes about a one second light stimulation to the prefrontal cortex. In some examples, the closed loop stimulation may include a pulse of light at about 2 mW to about 10 mW and about 10 Hz to about 30 Hz for about an about 1 ms to about 5 ms pulse width. In at least one example, the closed loop stimulation may include a pulse of light at 5 mW and about 20 Hz for about a 3 ms pulse width. In some examples, the light may be a blue light. The light may have a wavelength of about 473 nm.

In an embodiment, stimulator 150 may be configured to provide optical stimuli to the brain region. For example, the brain region may include photo-sensitive retinylidene proteins such as a channelrhodopsin. Channelrhodopsins may provide a controllable ion channel configured to actuate based on received light. Channelrhodopsin-1 (ChR1), Channelrhodopsin-2 (ChR2), or other variants are contemplated herein. Channelrhodopsins may provide sensitivity at various wavelengths of light. For example, the ChETA Channelrhodopsin may be opened by a blue light pulse and closed with a yellow light pulse. Such channelrhodopsins, other optically sensitive channel proteins, or other means for providing one or more neurons with sensitivity to optical stimuli could be provided to the brain region via a genetic therapy, e.g., by infection of cells in the brain region by a virus that includes genes coding for the channelrhodopsin or other proteins of other cell components. Other wavelength sensitive ion channels are possible.

Accordingly, stimulator 150 may include one or more light sources configured to actuate a channelrhodopsin or another type of light-sensitive ion channel. For example, stimulator 150 may include a blue laser diode coupled to a single-mode or multi-mode optical fiber, which may be delivered to the desired brain region. In an example embodiment, the blue laser diode may provide 1.6 mW/μm² at 473 nm wavelength. Stimulator 150 may also include various optical components, such as lenses, optical filters, and apertures, among other possibilities. As such, stimulator 150 may be configured to provide light at one or more wavelengths to the brain region so as to indirectly provide an excitatory electrical signal to cells of the brain region (via ion channels of the channelrhodopsin opening and closing).

Controller 150 may include a processor 152 and a memory 154, such as a non-transitory computer readable medium. Memory 154 may be configured to store instructions 156. Instructions 156 may be executed by processor 152 so as to carry out various operations described herein.

Processor 122 may include one or more general purpose processors—e.g., microprocessors—and/or one or more special purpose processors—e.g., image signal processors (ISPs), digital signal processors (DSPs), graphics processing units (GPUs), floating point units (FPUs), network processors, or application-specific integrated circuits (ASICs). Additionally or alternatively, the processor 122 may include at least one programmable in-circuit serial programming (ICSP) microcontroller. The memory 126 may include one or more volatile and/or non-volatile storage components, such as magnetic, optical, flash, or organic storage, and may be integrated in whole or in part with the processor 122. Memory 126 may include removable and/or non-removable components.

Processor 122 may be capable of executing program instructions (e.g., compiled or non-compiled program logic and/or machine code) stored in memory 126 to carry out the various functions described herein. Therefore, memory 126 may include a non-transitory computer-readable medium, having stored thereon program instructions that, upon execution by system 100, cause system 100 to carry out any of the methods, processes, or operations disclosed in this specification and/or the accompanying drawings. The execution of program instructions by processor 122 may result in processor 122 using data provided by various other elements of the system 100. Specifically, the controller 120 and the processor 122 may perform operations on based on information received from electrodes 110 and/or data acquisition system. In an example embodiment, the controller 120 may include a distributed computing network and/or a cloud computing network.

The controller 120 may be configured to operate the elements of the system 100 to detect electrical activity signals from a network of two or more regions of the brain, to determine a predicted time of onset of a neurological behavior, and to provide a stimulation signal to the brain prior to the predicted time of onset. Such operation could be performed to provide suppression of the neurological behavior. In some examples, the manifestation of the neurological behavior may be reduced or prevented as compared to the neurological behavior without providing the stimulation signal.

In an example embodiment, controller 120 may be configured to receive information indicative of an electrical activity signal from two or more brain regions in a network. Controller 120 may also be configured to determine, based on the received information, a predicted time of onset of the neurological behavior. Additionally, controller 120 may be configured to, in response to determining the predicted time of onset, send at least one stimulation signal to the brain. Yet further, controller 120 may be configured to, in response to the predicted time of onset, cause the stimulator 150 to provide stimulation to a brain region.

Additionally or alternatively, controller 120 may be configured to carry out some or all of the method steps or blocks described in relation to method 400.

Two or more of the elements of system 100 may be physically and/or communicatively coupled to one another via communication interface 130. Furthermore, communication interface 130 may allow system 100 to communicate, using analog or digital modulation, with other devices, access networks, and/or transport networks. Thus, communication interface 130 may facilitate circuit-switched and/or packet-switched communication, such as plain old telephone service (POTS) communication and/or Internet protocol (IP) or other packetized communication. For instance, communication interface 130 may include a chipset and antenna arranged for wireless communication with a radio access network or an access point. Also, communication interface 130 may take the form of or include a wireline interface, such as an Ethernet, Universal Serial Bus (USB), or High-Definition Multimedia Interface (HDMI) port. Communication interface 130 may also take the form of or include a wireless interface, such as a Wifi, BLUETOOTH®, global positioning system (GPS), or wide-area wireless interface (e.g., WiMAX or 3GPP Long-Term Evolution (LTE)). However, other forms of physical layer interfaces and other types of standard or proprietary communication protocols may be used over communication interface 130. Furthermore, communication interface 130 may comprise multiple physical communication interfaces (e.g., a Wifi interface, a BLUETOOTH® interface, and a wide-area wireless interface).

Power supply 140 may include one or more batteries. The batteries may include secondary (rechargeable) or primary (non-rechargeable) cells. Additionally or alternatively, at least some elements of system 100 may be powered by a conventional wall plug outlet (e.g., 120V, 60 Hz) or another type of energy source. In an example embodiment, power supply 140 includes an implantable battery that may be recharged via a wireless charging system.

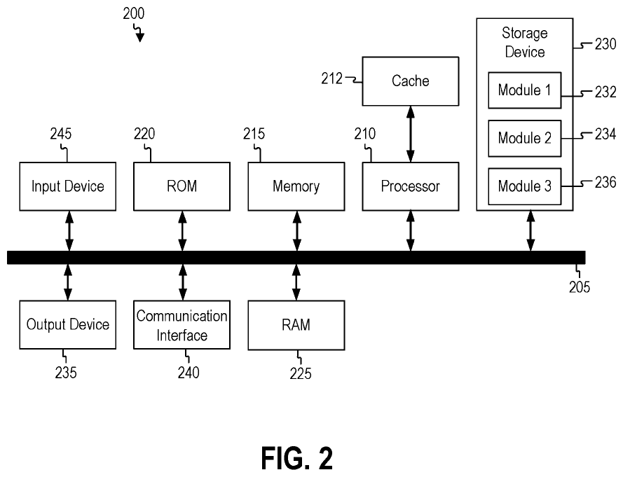

The disclosure now turns to another example system illustrated in FIG. 2 which may be used to implement the methods for predicting and treating a neurological behavior in a subject. FIG. 2 shows an example of computing system 200 in which the components of the system are in communication with each other using connection 205. Connection 205 can be a physical connection via a bus, or a direct connection into processor 210, such as in a chipset or system-on-chip architecture. Connection 205 can also be a virtual connection, networked connection, or logical connection.

In some examples computing system 200 is a distributed system in which the functions described in this disclosure can be distributed within a datacenter, multiple datacenters, a peer network, throughout layers of a fog network, etc. In some examples, one or more of the described system components represents many such components each performing some or all of the function for which the component is described. In some examples, the components can be physical or virtual devices.

Example system 200 includes at least one processing unit (CPU or processor) 210 and connection 105 that couples various system components including system memory 215, read only memory (ROM) 220 or random access memory (RAM) 225 to processor 210. Computing system 200 can include a cache of high-speed memory 212 connected directly with, in close proximity to, or integrated as part of processor 210.

Processor 210 can include any general purpose processor and a hardware service or software service, such as services 232, 234, and 236 stored in storage device 230, configured to control processor 210 as well as a special-purpose processor where software instructions are incorporated into the actual processor design. Processor 210 may essentially be a completely self-contained computing system, containing multiple cores or processors, a bus, memory controller, cache, etc. A multi-core processor may be symmetric or asymmetric.

To enable user interaction, computing system 200 includes an input device 245, which can represent any number of input mechanisms, such as a plurality of electrodes, a microphone for speech, a touch-sensitive screen for gesture or graphical input, keyboard, mouse, motion input, speech, etc. Computing system 200 can also include output device 235, which can be one or more of a number of output mechanisms known to those of skill in the art. In some instances, multimodal systems can enable a user to provide multiple types of input/output to communicate with computing system 200. Computing system 200 can include communications interface 240, which can generally govern and manage the user input and system output, and also connect computing system 200 to other nodes in a network. There is no restriction on operating on any particular hardware arrangement and therefore the basic features here may easily be substituted for improved hardware or firmware arrangements as they are developed.

Storage device 230 can be a non-volatile memory device and can be a hard disk or other types of computer readable media which can store data that are accessible by a computer, such as magnetic cassettes, flash memory cards, solid state memory devices, digital versatile disks, cartridges, battery backed random access memories (RAMs), read only memory (ROM), and/or some combination of these devices.

The storage device 230 can include software services, servers, services, etc., that when the code that defines such software is executed by the processor 210, it causes the system to perform a function. In some examples, a hardware service that performs a particular function can include the software component stored in a computer-readable medium in connection with the necessary hardware components, such as processor 210, connection 205, output device 235, etc., to carry out the function.

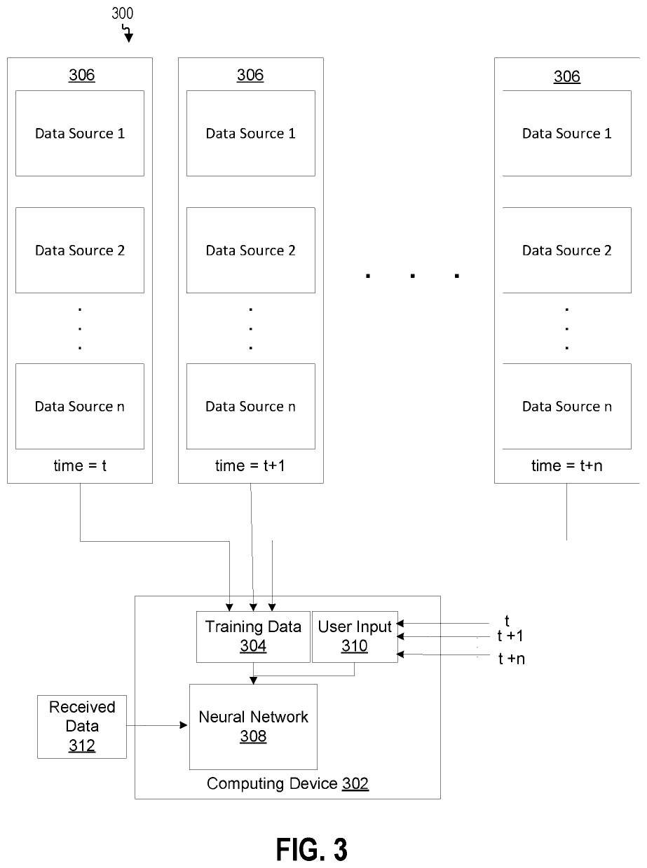

The disclosure now turns to FIG. 3, which illustrates an example machine learning environment 300. The machine learning environment can be implemented on one or more computing devices 302 (e.g., cloud computing servers, virtual services, distributed computing, one or more servers, etc.). The computing device(s) 302 can include training data 304 (e.g., one or more databases or data storage device, including cloud-based storage, storage networks, local storage, etc.). In some examples, the training data may include data from observed animals when engaged in different situations (e.g. attack behavior, non-attack behavior, appetitive social behavior, depressive behavior, etc.). The training data 304 of the computing device 302 can be populated by one or more data sources 306 (e.g., data source 1, data source 2, data source n, etc.) over a period of time (e.g., t, t+1, t+n, etc.). In some examples, training data 304 can be labeled data (e.g., one or more tags associated with the data). For example, training data can be electrical signals from one or more networks and a label (e.g., attack behavior, non-attack behavior, appetitive social behavior, depressive behavior, etc.). The computing device(s) 302 can continue to receive data from the one or more data sources 306 until the neural network 308 (e.g., convolution neural networks, deep convolution neural networks, artificial neural networks, learning algorithms, etc.) of the computing device(s) 302 are trained (e.g., have had sufficient unbiased data to respond to new incoming data requests and provided an autonomous or near autonomous prediction of onset of a neurological behavior). In some examples, the neural network can be a convolutional neural network, for example, utilizing five layer blocks, including convolutional blocks, convolutional layers, and fully connected layers. While example neural networks are realized, neural network 308 can be one or more neural networks of various types are not specifically limited to a single type of neural network or learning algorithm.

In some examples, while not shown here, the training data 304 can be checked for biases, for example, by checking the data source 306 (and corresponding user input) verse previously known unbiased data. Other techniques for checking data biases are also realized. The data sources can be any of the sources of data for providing the input electrical signals from a network of two or more brain regions as described above in this disclosure.

The computing device(s) 302 can receive user (e.g., physician) input 310 related to the data source. In some examples, the user input 310 and the data source 306 can be temporally related (e.g., by time t, t+1, t+n, etc.). That is, the user input 310 and the data source 306 can be synchronous in that the user input 310 corresponds and supplements the data source 306 in a manner of supervised or reinforced learning. For example, a data source 506 can provide an electrical signal from a network of brain regions of a subject at time t and corresponding user input 510 can be state of the subject (attack, non-attack, aggressive, non-aggressive, appetitive social behavior, depressive behavior, etc.) at time t. While time t may actually be different in real-world time, they may be synchronized in time with respect to the data provided to the training data. In other examples, the data source 306 may be used in a manner of unsupervised learning without user input.

The training data 304 can be used to train a neural network 308 or learning algorithms (e.g., convolutional neural network, artificial neural network, etc.). The neural network 308 can be trained, over a period of time, to automatically (e.g., autonomously) determine what the user input 310 would be, based only on received data 312 (e.g., signals from the plurality of electrodes, etc.). For example, by receiving a plurality of unbiased data and/or corresponding user input for a long enough period of time, the neural network will then be able to determine what the user input would be when provided with only the data. For example, a trained neural network 308 will be able to receive electrical activity signals from a network of two or more regions of the subject's brain from a plurality of electrodes (e.g., 312) and based on the electrical activity signals predict the onset of a neurological behavior that a physician would not be able to manually identify. In some examples, this can be based on labels associated with the data as described above.

Trained neural network (e.g. trained machine learning model) system 316 can include a trained neural network 308 and received data 312. The received data 312 can be information related to a subject/patient, as previously described above. The received data 312 can be used as input to the trained neural network 308. Trained neural network 308 can then, based on the received data 312, classify the received data and/or predict/determine a recommended course of action for treating the patient, based on how the neural network was trained (as described above). In some aspects, the output from the trained neural network can send a signal for treatment. For example, the output may be a stimulation signal sent to the brain. In other instances, the output from the trained neural network can be provided in a human readable form, for example, to be reviewed by a physician to determine a course of action.

For clarity of explanation, in some instances the present technology may be presented as including individual functional blocks including functional blocks comprising devices, device components, steps or routines in a method embodied in software, or combinations of hardware and software.

In some embodiments the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer readable media. Such instructions can comprise, for example, instructions and data which cause or otherwise configure a general purpose computer, special purpose computer, or special purpose processing device to perform a certain function or group of functions. Portions of computer resources used can be accessible over a network. The computer executable instructions may be, for example, binaries, intermediate format instruction ns such as assembly language, firmware, or source code. Examples of computer-readable media that may be used to store instructions, information used, and/or information created during methods according to described examples include magnetic or optical disks, flash memory, USB devices provided with non-volatile memory, networked storage devices, and so on.

Devices implementing methods according to these disclosures can comprise hardware, firmware and/or software, and can take any of a variety of form factors. Typical examples of such form factors include laptops, smart phones, small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

### III. Methods

Further provided herein are methods for predicting and treating a neurological behavior in a subject. The method can be performed automatically such that the prediction of the onset of the neurological behavior occurs in a timescale that allows for treatment of the neurological behavior by applying stimulation to the subject prior to the predicted onset of the neurological behavior. This can suppress or prevent the neurological behavior from manifesting.

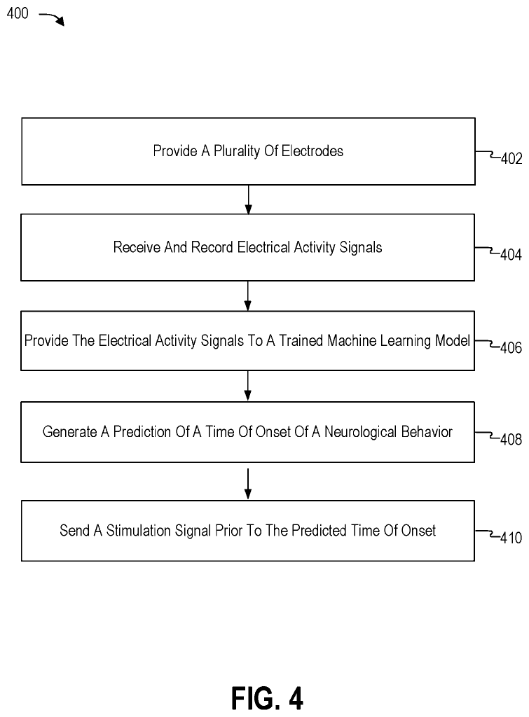

The overall framework of the method 400 for predicting and treating a neurological behavior in a subject is shown in FIG. 4. The neurological behavior may an aggressive state, an aggressive attack, an appetitive social behavior, or a depressive behavior.

At step 402, the method 400 can include providing a plurality of electrodes configured to receive electrical activity signals from a network of two or more regions of the subject's brain. In some examples, the plurality of electrodes are implanted in the subject's brain. The two or more regions of the brain in the network may include 2 to 10, 5 to 12, or 8 to 11 regions of the subject's brain. For example, the network may include 2, 3, 4, 5, 6, 7, 8, 9, 10, or 11 regions of the brain. Non-limiting examples of the two or more brain regions include infralimbic cortex, orbitofrontal cortex, prelimbic cortex, lateral septum, nucleus accumbens, lateral habenula, mediodorsal thalamus, ventromedial hypothalamus, medial amygdala, primary visual cortex, ventral hippocampus, and combinations thereof.

At step 404, the method 400 can include receiving and recording the electrical activity signals from the plurality of electrodes, via a controller in communication with the plurality of electrodes.

At step 406, the method 400 can optionally include providing the electrical activity signals to a trained machine learning model. In other examples, the method 400 does not utilize a trained machine learning model. Instead, the method 400 may include comparing the electrical activity signals to a threshold.

At step 408, the method 400 can include generating a prediction of a time of onset of the neurological behavior. In some examples, the prediction is generated from the trained machine learning model. In other examples, the prediction is generated when the electrical activity signals decrease below the threshold. Once the signals are below the threshold, a prediction of a time of onset of the neurological behavior can be generated. The threshold can be determined from a baseline of electrical activity signals from the network in the absence of the neurological behavior. A predicted time of onset of the neurological behavior can be determined from the reduction in the network electrical signal. In additional examples, the electrical activity signals can be provided to a trained machine learning model that generates a prediction of a time of onset of the neurological behavior from the trained machine learning model. In various examples, the predicted time of onset of the neurological behavior is on a second timescale. For example, the predicted time of onset may be provided about 1 s, about 2 s, about 3 s, about 4 s, about 5 s, about 6 s, about 7 s, about 8 s, about 9 s, or about 10 s before the neurological behavior manifests.

At step 410, the method 400 can include sending a stimulation signal to the brain prior to the predicted time of onset of the neurological behavior. The stimulation signal to the brain suppresses or prevents the neurological behavior. In some examples, the stimulation signal may be sent to the desired brain region about 1 s, about 2 s, about 3 s, about 4 s, about 5 s, about 6 s, about 7 s, about 8 s, about 9 s, or about 10 s before the predicted onset of the neurological behavior. The desired brain region for the stimulation signal may be the same or different than one or more of the brain regions in the network. In some examples, the stimulation signal is directed to the prefrontal cortex of the subject's brain. The stimulation signal can be a light signal as described herein. For example, the stimulation of the desired brain region can include closed loop optogenetic stimulation.

The methods, systems, and devices described herein could be used to provide a therapy to a person, e.g., to alleviate the symptoms and/or causes of one or more neurological or psychological conditions or disorders. For example, the methods, systems, and devices described herein could be used to aggression, treat bipolar disorder or other mood disorders, post-traumatic stress disorder or other anxiety disorders, schizophrenia and/or memory loss, autism, or other psychological conditions or disorders.

Such therapies could be provided in response to a diagnosis that a patient is experiencing one or more relevant psychological disorders or conditions. Further, such therapies could be provided following the application of other therapies having incomplete success or effectively in treating the causes, symptoms, or other aspects of a diagnosed psychological condition. For example, if providing the therapy is predicated by the implantation of one or more devices or electrodes (e.g., a cylindrical lead to access deep structures within the brain and/or penetrating or surface elements to access surface structures of the brain, along with any associated leads, implanted controllers, transcutaneous connectors, or other implanted elements), less invasive therapies (e.g., talk therapy, pharmaceutical interventions) may be pursued before applying therapies as described herein.

Providing such a therapy could include detecting electrical activity signals in a network of two or more brain regions and providing, to a desired brain region, one or more stimulation signals prior to a predicted onset time for a behavior that is desired to be suppressed. Electrical activity signals (oscillatory signals) can be detected from a number of different brain regions and/or stimulation signals based on such detected signals could be provided to multiple different brain regions.

The therapy could be provided chronically (e.g., continuously or as a number of discrete therapies over months or years) or as an acute therapy (e.g., one or more discrete sessions). An acute therapy, or discrete instances of a chronic therapy, could be provided by devices that are present in a physician's office or hospital. For example, electrical activity signals could be detected using an array of EEG electrodes, a magnetoencephalogram, a magnetic resonance imager, or other devices or systems. Conversely, excitatory stimuli could be provided by transcranial magnetic stimulation. Additionally or alternatively, aspects of an acute therapy (e.g., detection of an oscillatory signal and/or providing excitatory stimuli) could be provided by implanted devices that may be removed and/or disabled following completion of the therapy. An acute therapy could be provided for a specified period of time, number of sessions, or amount of deliver stimulation. Additionally or alternatively, an acute therapy could be provided until to condition is detected, e.g., until a specified degree of suppression of the neurological behavior, until a specified degree of alleviation of signs or symptoms of the neurological behavior has been observed, or until some other condition has been met.

Providing such a therapy could include implanting one or more devices. For example, providing such a therapy could include implantation of sub-dural or supra-dural electrodes, surface electrodes, penetrating electrodes or optical fibers, leads, controllers, or other implanted elements of a device or system. Some of these elements could be implanted stereotaxically, e.g., to facilitate the positioning of stimulating means and/or signal-detecting means in a target brain region. Implantation of such elements could include performance of test stimulation and/or recordings to verify the accurate placement of such elements intra-operatively.

Such a therapy could include determining settings for provision of the therapy. Such settings could include amplitudes, dosages, pulse waveforms, frequencies, or other properties of provided stimulus. Such settings could include determining a relative timing between a predicted onset of the neurological behavior and the onset of a provided stimulus/stimulation signal (e.g., to maximize a positive effect of the stimulus on one or more brain regions). The determination of such settings can be automated (e.g., based on one or more detected signals applied to a trained machine learning model, an algorithm, or some other automated method of parameter determination).

Providing such a therapy can include providing adjunct therapies in combination with the provision of the stimulation signal. For example, pharmaceuticals could be provided in addition to such stimulation. The amount of such pharmaceuticals provided to a patient could be decreased as the effects of the stimulation increase.

### IV. Examples

Social aggression is an innate behavior that can aid an organism in securing access to resources. Aggression can also disrupt group function and reduce survival under conditions of behavioral pathology. Since many brain regions contribute to multiple social behaviors, expanded knowledge of how the brain distinguishes between social states would enable the development of interventions that suppress aggression, while leaving other social behaviors intact. Here it was shown that a murine aggressive internal state is encoded by a brain-wide network. This network is organized by prominent theta (4-11 Hz) and beta (14-30 Hz) oscillations, leading from orbital frontal cortex and medial dorsal thalamus, and converging on ventral medial hypothalamus and medial amygdala. Activity in this network couples to brain-wide cellular firing, and the network is conserved in multiple contexts associated with aggression. Strikingly, network activity during social isolation encodes the trait aggressiveness of mice and causal cellular manipulations known to impact aggression bidirectionally regulate network activity. Finally, it was shown that the network plays a role in mediating aggressive behavior through causal mediation analysis. Thus, a brain-wide network was defined that encodes an aggressive internal state within and across mice.

Social behavior reflects the integration of sensory information with internal affective states. Many subcortical brain regions contribute to aggressive behavior in mammals including lateral septum (LSN), nucleus accumbens, lateral habenula, the ventrolateral portion of ventromedial hypothalamus, and medial amygdala. Prefrontal cortex stimulation has been shown to mitigate aggressive behavior in both humans and rodents, implicating cortical regions in regulating aggression. Finally, sensory regions, such as those underlying olfaction, also contribute to aggression.

To appropriately regulate aggressive behavior, the brain must integrate information across these and other cortical and subcortical regions. Moreover, since many of these regions also regulate non-aggressive social behaviors, the brain must ultimately utilize information from overlapping regions to segregate aggression from other social behavioral states. Though efforts have revealed several cellular-level processes within regions that contribute to this mechanism, the complementary network level process that integrates information across regions to distinguish aggressive states from prosocial states remains unknown. Addressing this question is of major importance as 1) mammals regularly select from a repertoire of social behaviors based on external sensory cues to ensure their survival, and 2) a range of psychiatric disorders are broadly marked by a failure to appropriately match behavior with evolving social contexts.

Mice were implanted with microwire electrodes across eleven brain regions implicated in regulating complex social behavior. Electrical activity was then recorded from these brain regions, concurrently, as mice engaged in social encounters that induced aggressive attack behavior and non-attack social behavior. After confirming that statistical models based on single brain regions could independently differentiate attack behavior from non-attack social behaviors, it was asked whether this code was also reflected at a network level, where millisecond timescale information was integrated across all the brain areas. For this analysis, a machine learning approach was used that models variations in natural patterns of activity within and between implanted brain regions across seconds of time (a timescale that we reasoned would allow us to capture socially-relevant internal states). This approach also tuned the model to optimally encode attack vs. non-attack social behavior.

Multiple levels of validation of increasing generalization were then applied to the single region and network level models. 1) It was tested whether the models generalized to data collected from time periods that had not been used to train the model (i.e., hold out sessions). 2) It was tested whether the models generalized to new animals, and 3) It was tested an orthogonal behavioral context associated with aggression. Only the model that was based solely on activity from ventral hippocampus and the network model survived this level of validation. 4) Next, it was tested whether these two models encoded an aggressive state, more broadly, and not simply attack behavior. Only the network model encoded the social context of mice during periods immediately prior to or following aggressive and prosocial interactions. Moreover, network activity during this interval correlated with trait aggressiveness on a mouse-by-mouse basis. 4) Finally, the network was subjected to two high levels of experimental validation, each based on causal cellular manipulations, that bidirectionally impact attack behavior. Specifically, a closed loop optogenetic stimulation approach was developed that detected when the brain transitioned into an aggressive state, on a second-by-second basis, and stimulated the prefrontal cortex in a manner previously known to suppress attack behavior. A designer receptor exclusively activated by designer drug (DREADD) based approach was employed to selectively activate cells in ventral medial hypothalamus in a manner that has been shown to induce attack behavior. These manipulations bidirectionally impacted network activity. 5) As this latter manipulation was also performed in mice on a different genetic background, it was further confirmed the generalizability of the network-level findings. Thus, a network-level mechanism was established whereby the brain encodes an aggressive state in a manner that generalizes across context and individuals.

Animals care and use

Six-month retired breeder male CD1 strain mice were used to discover a network that encoded aggression, hereafter called EN-AggINH. These CD1 mice were also used to probe the behavioral and network responses to optogenetic stimulation. Mice were singly housed with enrichment. ESR1-Cre mice on a C57/B16J background were provided by Scott Russo. These mice were crossed with CD1 females in the Bryan Vivarium at Duke University to obtain F1 offspring. Fourteen-week-old virgin ESR1-cre F1 male offspring were used to validate EN-AggINH. All F1 offspring were group-housed 2-5 mice per cage until they received viral injections in the ventromedial hypothalamus at 7-8 weeks. After surgery, these mice were singly housed with enrichment. All partner mice (intact males, female, and castrated males) were 7-14 weeks old. These mice were maintained on a C57BL/6J strain background. All stimulus mice were housed 5 per cage with enrichment. All behavior testing and neurophysiological recordings occurred during the dark cycle.

### Electrode Implantation Surgery

Mice were anesthetized with 1% isoflurane and placed in a stereotaxic device. Grounding screws were placed above the cerebellum, right parietal hemisphere, and anterior cranium. The recording bundles were designed to target prelimbic cortex, infralimbic cortex, medial amygdala, ventral hippocampus, primary visual cortex, mediodorsal thalamus, lateral habenula, lateral septum nucleus, nucleus accumbens, ventrolateral portion of the ventromedial hypothalamus, and orbitofrontal cortex were centered based on stereotaxic coordinates measured from bregma. [Orbital frontal cortex: anterior/posterior (AP) 2.35 mm, medial/lateral (ML) 1.0 mm, dorsal/ventral (DV) from dura-2.75 mm; infralimbic cortex and prelimbic cortex: AP 1.8 mm, ML 0 mm, DV −2.7 mm from dura; medial amygdala: AP-1.25, ML 2.7 mm, DV −4.3 from dura; lateral septum and nucleus accumbens: AP 1.0 mm, ML 0 mm, DV −4.0 mm from dura; ventromedial hypothalamus, lateral habenula, and medial dorsal thalamus: AP-1.47 mm, ML 0 mm, DV −5.4 mm from dura; central hippocampus and primary motor cortex: AP-3.0 mm, ML 2.6 mm, DV −3.0 mm from dura]. Infralimbic cortex and prelimbic cortex were targeted by building a 0.6 mm DV stagger into the bundle. We targeted lateral septum and nucleus accumbens by building a 0.3 mm ML and 1.5 mm DV stagger into the bundle. Lateral habenula, medial dorsal thalamus, and ventral medial hypothalamus were targeted by building a 0.3 mm ML and 1.9 mm and 2.5 mm DV stagger into our electrode bundle microwires. Primary motor cortex and ventral hippocampus were targeted using a 0.3 mm ML and 2.5 mm DV stagger in our electrode bundle microwires. For optogenetic stimulation experiments, the addition of a Mono Fiberoptic Cannula coupled to a 2.5 mm metal ferrule (NA: 0.22, 100 mm [inner diameter], 125 mm buffer [outer diameter], MFC_100/125-0.22,) was built into the prefrontal cortex bundle. The tip of the fiber was secured 300 mm above the tip of the IL microwire. Mice were allowed 10-15 days for to recovery from surgery before behavioral testing.

### Viral Surgery

For optogenetic experiments 21, we used CD1 mice that showed an attack latency <60s when exposed to an intact C57 male. CD1 mice were anesthetized with 1% isoflurane and placed in a stereotaxic device. The mice were unilaterally injected with AAV2-CamKII-Chr2-EYFP, based on stereotaxic coordinates from bregma (left Infralimbic cortex: AP 1.8 mm, ML 0.3 mm, DV −2.0 mm from the brain). A total of 0.5 mL of virus was infused at the injection site at a rate of 0.1 mL/min over five minutes and the needle left in place for ten minutes after injection. For the open loop stimulation experiment, CD1 mice were implanted with an optic fiber (Mono Fiberoptic Cannula coupled to a 2.5 mm metal ferrule (NA: 0.22, 100 mm [inner diameter], 125 mm buffer [outer diameter], MFC_100/125-0.22)) 0.3 mm above the injection site immediately after viral syringe was removed. These mice were allowed 3 weeks for recovery prior to behavioral testing. For the closed-loop experiments, CD1 mice were allowed 3 weeks for viral expression prior to implantation with an optrode.

For the ESR1-Cre validation experiment, F1 offspring were bilaterally injected with AAV2-hSyn-DIO-GqDREADD based on stereotaxic coordinates measured from bregma (AP-1.5 mm, ML+0.7 mm, DV −5.7 mm from the dura). A total of 0.3 mL of virus was infused bilaterally at a rate of 0.1 mL/min and the needle left in place for five minutes after injection. Two weeks after viral infusion, F1 males were screened for aggressive behavior towards females. The F1 males received i.p. injections of CNO (1 mg/kg) at the start of the screening session. Thirty-five minutes after injection, a novel C57 female was placed in the home cage for 5 minutes. Screening was repeated one week and two weeks later. Only F1 males who attacked females for at least two of the three screening sessions (9/13 mice) were implanted with electrodes 17. The eight mice that showed good surgical recovery were subjected to further experiments.

### Histological Analysis

Histological analysis of implantation sites was performed at the conclusion of experiments to confirm recording sites and viral expression. Animals were perfused with 4% paraformaldehyde (PFA) and brains were harvested and stored for at least 96 hrs in PFA. Brains were cryoprotected with sucrose and frozen in OCT compound and stored at −80C. Brains were later sliced at 40 mm. Brains from mice used to train and validate the network were stained using NeuroTrace fluorescent Nissl Stain using standard protocol. Specifically, Nissl staining for brain tissue occurred on a shaker table at room temperature. Tissue was washed in PBST (0.1% Triton in phosphate-buffered saline solution) for 10 minutes. It was then washed for five minutes in PBS twice. The tissue was then protected from light for the remainder of the protocol. The tissue was incubated in 1:300 Nissl diluted in 2 mL PBS for 10 minutes. After the Nissl incubation, tissue was washed once in 0.1% PBST for 10 minutes, then twice in PBS for 5 minutes. Brains from ESR1 mice and mice used for 20 Hz or closed loop stimulation were mounted in Vectashield mounting medium containing DAPI. Images were obtained at 10× using an Olympus fluorescent microscope. Of the 297 total implantation sites in the training and testing set of mice, 17 were mistargeted (~5.7% error rate). Of these mistargeted implants, 13 were within 200 μm of the targeted structure. Given our prior work demonstrating high LFP spectral coherence (in the 1-55 Hz frequency range) across microwires separated by 250 μm, in both cortical and subcortical brain regions, it was chosen to retain these animals in our analysis. The other four mistargeted implants were within 350 μm of the targeted structure. The most reliably mistargeted site was ventral medial hypothalamus for which 4 animals were implanted within 200 μm of the target, and 2 animals were implanted within 350 μm of the target.

Machine learning analysis typically benefits from larger data sets. Thus, maintaining a higher number of data points likely outweighed the effect of a small number of mistargeted brain regions, particularly since the LFP measures were robust to the targeting inaccuracies observed histologically. As such, data from all 20 implanted animals was pooled to learn our initial model. A similar strategy was employed for the validation analysis, where an animal was only removed from the validation set if there was clear histological confirmation of mistargeting >200 μm for any of the recorded regions. Specifically, presuming accurate targeting with 94.3% certainty and targeting within 200 μm at a higher certainty, we included animals with missing or damaged histological slices in our analysis. However, if there was clear histological confirmation of mistargeting for any of the recorded regions, the animal was removed from the validation testing. Critically, our validation procedure implies that the machine learning models were robust regardless of any slight imprecision in the animals we utilized for training.

### Neurophysiological Data Acquisition

Mice were connected to a data acquisition system while anesthetized with 1% isoflurane. Mice were allowed 60 minutes in their home cage prior to behavioral and electrophysiological recordings. Local field potentials (LFPs) were bandpass filtered at 0.5-250 Hz and stored at 1000 Hz. An online noise cancellation algorithm was applied to reduce 60 Hz artifact. Neural spiking data was referenced online against a channel recording from the same brain area that did not exhibit a SNR>3:1. After recording, cells were sorted using an offline sorting algorithm to confirm the quality of the recorded cells. Only cell clusters well-isolated compared to background noise, defined as a Mahanalobis distance greater than 3 compared to the origin, were used for the unit-Electome Factor correlation analysis. We used both single and multi-units for our analysis as our objective was to determine whether Electome Network activity was reflective of cellular activity. Neurophysiological recordings were referenced to a ground wire connected to ground screws above the cerebellum and anterior cranium.

### Behavioral Recordings and Analysis for Training/Testing Models

The CD1 mice used for training and testing the electome model were first subjected to screening to assess their basal level of aggressiveness. Screening occurred once a day for three consecutive days prior to surgical implantation. For each screening session, an intact male C57 was placed in the CD1's home cage for 5 minutes and the latency to first attack was recorded. To ensure that our network generalized broadly across CD1 mice, a training and testing set was used for which ~50% of the mice showed high aggression (i.e., latency to attack <60s), and ~50% of the mice showed low to moderate aggression (i.e., latency to attack >60s). Animals were screened in cohorts, and we excluded 16/45 mice from further experiments.

All screening/testing occurred within the home cage of mice except for the quantification of cortical stimulation induced gross locomotor activity. These latter experiments were performed in a 44 cm×44 cm×35 cm (L×W×H) open field arena. Subject mice (CD1 and ESR1 males) were acclimated to the recording tether for three days prior to the first recording session. Each acclimation session involved anesthetizing the mouse with 1% isoflurane, tethering the subject mouse, allowing 60 minutes to recover from isoflurane, then placing a male C57 in the home cage for 5 minutes. Mice were then anesthetized with isoflurane again and detached from the tether. The aggressive tone of experimental mice was determined based on average latency to attack partner mice during the second and third acclimation sessions.

After screening, mice were implanted, and data was acquired across 1-6 behavioral testing/recording sessions following recovery. Sessions were separated by 5-7 days. Recordings for all social encounters were performed in the home cages of the CD1 mice. Each behavioral testing session began with 5 minutes of recording prior to introduction of the social stimulus. All mice were subjected to encounters with an intact C57 male mouse and a female c57 mouse. A subset of eighteen mice were also subjected to an encounter with a castrated male mouse, and another subset of eighteen mice were subjected to exposure to objects covered in CD1 mouse urine. Six mice were recorded under all four conditions. Data observations (1 second each) were pooled across eleven CD1 mice for training the network model. Object pairs included yellow duplex blocks, curved red duplex blocks, weighted 5 ml conicals, glassware tops, and objects assembled from black Legos®. The CD1 mice were exposed to a different pair of objects during each session. Order of exposure to stimulus mice and objects was shuffled for every session.

For ESR1 male behavioral testing, mice were injected with either saline or CNO (1 mg/kg, i.p.) after the five-minute baseline recording. Thirty-five minutes after this injection, mice were exposed to an intact male C57, a castrated male C57, and a female C57, presented in pseudorandom order. Mice were subjected to six total recording sessions (three in which they were treated with saline and three in which they were treated with CNO), again in pseudorandom order. Sessions were separated by 5 days to allow an adequate washout of CNO46.

Behavior was scored for each second as an “attack”, “non-attack social interaction”, or “non-interaction”. One second windows were identified as “aggressive” if the mouse was engaged in biting, boxing (kicking/clawing), or tussling behavior. Windows were labeled as “non-attack social interaction” if the mouse had his nose or forepaws touching the stimulus mouse (intact male/female/castrated male) or object, but was not biting, boxing, or tussling. Examples of behaviors labeled “non-attack social behavior” included sniffing, grooming, or resting (placing nose or forepaws against the subject mouse, but not moving). If the stimulus mouse had his/her forepaws or nose on the CD1, but it was not reciprocated, this was labeled “non-interaction”. CD1 straight approach, sideways approach, and chasing of the stimulus mouse could result in attack (biting/kicking/tousling), non-attack social behavior (nose or paw touch), or withdrawal without any touch. Thus, while sideways approach and chasing are regularly labeled as “aggressive”, and straight approach is regularly labeled as “pro-social”, these behaviors lacked consistent resolutions. Moreover, mice also demonstrated these behaviors towards female and castrated mice (non-attack social context). One second windows containing these behaviors were labeled “non-interaction”. All other timepoints not labeled “attack” or “non-attack social” were also labeled “non-interaction”. These behavioral criteria were selected to include ethologically aggression-related behaviors and maximize the likelihood that the CD1 was aware of the presence of the stimulus mouse or object during the behavioral window, while remaining confident in the classification of “attack” and “non-attack social” window labels.

While tail rattling is not an attack behavior like the other behaviors that were labeled as “attack”, it was consistently only demonstrated by aggressive mice towards intact male mice. Moreover, tail rattling is well-recognized in the literature as an aggressive behavior. Thus, this behavior was included in the “attack behavior category.” In the subset of 20 mice used for training the network, tail rattling was observed 8±4s out of the 135±26s ‘attack windows’ per mouse.

The videos used to generate the labels for training and testing our machine learning model was hand-scored by a trained researcher. Videos from ESR1 mice and optogenetic stimulation were automatically tracked using DeepLabCut. This information was then used for creating behavioral classifiers in SimBA.

### LFP Preprocessing and Signal Artifact Removal

Each LFP signal was segmented into Is non-overlapping windows. If there were multiple intact channels implanted in a region, they were averaged to produce a single signal. Windows with non-physiological noise were removed using an established automated heuristic 7. Briefly, the envelope of the signal in each channel was estimated using the magnitude of the Hilbert transform. The Median Absolute Deviation (MAD) of the magnitude was then calculated on each channel of each recording. Signal was marked as non-physiological if the envelope exceeded a high threshold (5× MAD, which is roughly 4x the standard deviation for a normally distributed signal). Any data adjacent to non-physiological data that had an envelope value above a smaller threshold (0.167 MAD) was also considered non-physiological. All data marked in this way was ignored when averaging channels for each region. Any channels with standard deviation less than 0.01 was removed as well. If no channels were usable for a given region within a window, that whole window was removed from the data. This set of heuristics resulted in 34.7=5.1% of the data being excluded from analysis. After this, 60 Hz line artifact was further removed using a series of Butterworth bandpass filters at 60 Hz and harmonics up to 240 Hz with a stopband width of 5 Hz and stopband attenuation of −60 dB. Finally, the signal was downsampled to 500 Hz.

### Estimation of LFP Oscillatory Power, Cross-Spectral Coherence, and Granger Directionality.

Signal processing was performed using Matlab. For LFP power, spectral power was estimated using Welch's method using a 250-millisecond window and 125 millisecond step. Windows were zero-padded to give a 1 Hz resolution. Cross-spectral coherence was estimated pairwise between all regions using Welch's method and magnitude-squared coherence defined as

$${C}_{A B} ( ω ) = \frac{{\vert Ps {d}_{A B} ( ω ) \vert}^{2}}{P s {d}_{A A} ( ω ) P s {d}_{B B} ( ω )} ,$$

where A and B are two regions and Psd_{AA}(ω) and Psd_{AB}(ω) are the power and cross spectra at a given frequency ω, respectively.

Spectral Granger causality features were estimated using the Multivariate Granger Causality (MVGC) MATLAB toolbox. To get stable Granger causality estimates, a 6th order highpass Butterworth filter—with a stopband at 1 Hz and a passband starting at 4 Hz—was applied to the data using the filtfilt function (MATLAB, The Math Works, Inc. Natick MA). Granger causality values for each window were estimated with a 20 order AR model at 1 Hz intervals to align with the power and coherence features. Granger features were processed identically to a previously reported approach. Briefly, Granger features were exponentiated to approximately maintain the additivity assumption made implicitly by NMF models as, exp(f_{A→B}(ω)), where f_{A→B}(ω) is the Granger causality at frequency ω from region A to region B. The exponentiated feature is a ratio of total power to unexplained power. Exponentiated Granger feature values were truncated at 10 to prevent implausible values.

### Data for Single Region and Network-Level Machine Learning Analyses

21,460 seconds of data were used, pooled across the twenty mice, to train/validate our single region and network models. This included a total of 4680 seconds while mice were socially isolated in their home cage, 14,890 seconds where CD1 mice exhibited non-attack social behavior (3542 seconds towards intact males, 9067 seconds towards females, and 2,281 seconds towards castrated), and 1,890 seconds where mice exhibited attack behavior towards the intact males.

### Single Region Decoding

To test the efficacy of any single brain region as a biomarker for aggression, power was extracted at 1 Hz frequency bins over 1-56 Hz from each region. The fitglm function (MATLAB, The Math Works, Inc. Natick MA) was used for binary outcomes with the logit link function to build a predictive model from the power across frequencies. One second windows were pooled from the twenty CD1 mice and used to generate a series of logistic models for each of the 11 brain regions. The models were trained to distinguish behavioral windows from one social state exhibited by CD1 mice, from two other social states. These three social states included 1) male-directed attack, 2) female non-attack social interactions, and 3) castrated male non-attack social interactions. A model was established to distinguish 4) periods where CD1 were isolated in their home cage from any of the three social states. Each model was then tested on data from a holdout set of nine mice. The Area under the receiver operating curve (AUC) was calculated for each holdout mouse to determine the performance of the model. False discovery rate was used to correct for multiple hypothesis testing.

### Discriminative Cross-Spectral Factor Analysis-Nonnegative Matrix Factorization

The network was trained to distinguish between behavioral windows when the CD1 mice showed aggressive behavior towards intact C57 males, and windows where they exhibited pro-social behavior. These latter windows comprised pro-social interactions towards intact C57 males, castrated C57 males, or C57 females. Here, we used data from twenty-seven mice to learn the final model, with a split of 20 and 7 for model training and internal validation.

Discriminative Cross-Spectral Factor Analysis-Nonnegative Matrix Factorization (dCSFA-NMF) model was used. This approach assumes each window of is an independent stationary observation and examines dynamics in brain activity only at the scale of windows. A 1 second window was chosen to balance capturing fine-grained transient behavior with sufficient length to properly estimate spectral features. Each window has associated spectral power, coherence, and Granger causality features (in total p=9,586 features), which is represented as xᵢ∈ [symbol] ^{p} for the i^{th} window. Each window was associated with a behavioral label that identified which condition the CD1 mouse was subjected to during that window (intact male, castrated male, or female) and whether the CD1 mouse was engaged in aggressive or non-aggressive behavior during that window, and the aggressive behavior was coded as yᵢ∈{0,1}.

As a short description of the dCSFA-NMF model, the features are described as an additive positive sum of K non-nonnegative electrical functional connectome networks. This model is learned using a supervised autoencoder. The objective we use to learn the parameters is

$${min}_{W , d , ϕ} {\sum }_{i = 1}^{N} {Loss}_{X} ( {x}_{i} , Wf ( {x}_{i} ; ϕ ) ) + λ {Loss}_{Y} ( {y}_{i} , df ( {x}_{i} ; ϕ ) ) + μ {Loss}_{EN} ( A ) .$$

Here, Loss_{X} is the reconstruction loss of the features derived from electrophysiology, which for our work was an L₂ loss. Our predictive loss Lossy is the cross-entropy loss commonly used for binary classification. Each of the K networks is represented in vector form and combined to make a matrix W∈R^{p×K} The electome factor scores are given by the multi-output function f(x; φ): R^{p}→R^{K}, where φ represent the parameters of the function. In our model, the multi-output function was an affine transformation of Ax+b followed by a softplus rectification, defined as softplus(x)=log(1+exp x), thus φ={A, b}. Parameters d∈R^{k} represent the relationship between the electome factors and the behavior. A sparsity constraint is enforced so that d=[d₁, 0, . . . ,0], meaning that only a single electome is used to predict behavior, simplifying interpretation. λ is a weighting parameter used to control the relative importance of prediction. We chose a value that kept the two losses approximately equal during training, which corresponded to 1.

Previous work has also found that the reconstruction loss can reduce overfitting and make the learned predictions more robust. To further reduce overfitting of the predictive aspect of the encoder, an elastic net loss was applied on the encoder Loss_EN with a weighting u and the ratio between the L_1 and L_2 losses set to 0.5. The value for u was set to a small value that had worked well previously. In this work, power features were also upweighted by a factor of 10 to accommodate that there were many more Granger features and truncated at 6 to prevent outliers from dominating the predictions.

These models and statistical analyses were implemented with Python 3.7 and Tensorflow version 2.4. Parameters were learned with stochastic gradient descent using the Nesterov accelerated ADAM optimizer. Learning was performed for 30000 iterations, which was observed to be ample for parameter convergence. The learning rate and batch size were set to le-3 and 100 respectively, values that have empirically performed well in similar applications.

Predictive performance was evaluated in new mice not involved in learning the network. Given processed data from the new mice, factor scores were estimated as an evaluation of the encoder learned during training of the dCSFA-NMF model.

### Hyper-Parameter Selection

The dCSFA-NMF procedure requires selection of several settings in the algorithm. Specifically, we must choose the number of electome factors K, the importance of the supervised task 2, the relative importance of the power features, coherence features, and Granger features, and the parameterization of the mapping function f(xᵢ; ¢). Besides K, these settings were chosen to match previously used values or follow heuristics.

To choose the value of K, the reconstruction error (Mean Squared Error) on the seven internal validation mice was evaluated, which evaluates how well the Electome networks describe the neural measurements. As the goal for the analysis was to maintain high reconstruction and effectively predict the behavior, an elbow analysis was used to choose the number of electome networks K after which we observed minimal gains in explaining the data.

### Validating Model Dimensionality

A frequent concern of latent variable models (including dCSFA-NMF) is the dependence of the networks and encoder on the choice of latent dimensionality. To address this concern, we performed a sensitivity analysis on the supervised network to determine the extent to which the choice of this dimensionality influenced the learned aggressive network electome and encoder. In this sensitivity analysis, a dCSFA-NMF model was estimated allowing the number of total networks to range from two to twenty. The similarity between each learned encoder and decoder was compared to the model with eight networks (the final model used in this work). This was quantified using the cosine similarity, which measures the angle between two networks (or encoders), ranging from −1 to 1. A value of 1 indicates perfect alignment (pointing in the same direction), 0 is orthogonal, and −1 indicates that the vectors point in opposite directions.

It was found that the supervised network maintained a strong consistency across most dimensions, particularly between 5-10 networks, as shown by the cosine similarities being greater than 0.95. The supervised encoders were virtually identical all the models except the one that utilized three networks. This model learned a network that was positively associated with aggression.

To evaluate the robustness of the similarities across most of the supervised networks, a null distribution of the similarities across randomly chosen generative networks was created. These later similarities were substantially lower both for the network composition and the encoder. This indicates that as far as the supervised electome and encoder are concerned, latent dimensionality is not particularly influential on the resulting network, and by extension the biological interpretation.

### Decoder Information Content

The amount of information contained in the predictive model was quantified by the reduction in uncertainty. The associated formula for this reduction in uncertainty, known as the Bernoulli entropy, is 1-1*(p log_2(p)+(1−p)log_2 (1−p)), where p is the accuracy of the model. At the extremes, an accuracy of 0.5 (random guessing) removes no uncertainty, whereas an accuracy of 1 or 0 completely eliminates uncertainty.

### Single Cell Correlation to Electome Factor Activity

Data acquired during the third behavioral testing session was from the twenty implanted mice were used for cellular analysis. We used Spearman correlation to quantify the relationship between cellular firing windows and the activity of the electome network used to classify attack behavior. 1000 permutations were performed of randomly shuffling 1 second windows within each class for attack and non-attack social interactions with male, female and castrated C57 mice. This approach maintained the relationship between network activity and behavior and the relationship between cell firing and behavior. The spearman correlation between network activity and cell firing was then calculated for each permutation. A cell was deemed positively correlated if its unshuffled spearman Rho was above 97.5% of the permutated distribution and negatively correlated if it was below 2.5%.

### Real-Time Encoder Approximation

Because Granger causality features were too computationally demanding for real-time calculation, we developed a ‘fast’ dCSFA-NMF model that relied only on power and coherence features for estimation of aggressive state to use in the closed-loop stimulation experiments. This ‘fast’ model was trained on the same data. The model was trained using regularized regression to best predict the output of the full encoder. As such, this reduced encoder is also an affine transformation followed by a SoftPlus activation with a smaller parameter set, φᵣ={Aᵣ, bᵣ}. This approximation explained a large component of the variance of the supervised network score on the hold-out validation mice (R-0.47, p-value <10-16).

### Optogenetic Stimulation

Mice were anesthetized with 1% isoflurane, then tethered to for electrophysiological recording and had an optic patch cable places over the optic fiber cannula. The mice were then allowed 60 minutes for recovery prior to session recording. For the fiber only optogenetic stimulation experiments, CD1 mice experienced two stimulation sessions. For closed loop optogenetic stimulation, CD1 mice experienced three sessions of behavioral screening, then two sessions of closed-loop, then two sessions of open-loop, then one session of random stimulation. Stimulation sessions were separated by 5-7 day between sessions. For behavioral screening, CD1 mice were exposed to intact C57 males, females, and castrated male mice for 5 minutes each. Screening session two and three were used to determine reduced network threshold at which 40% of aggressive behavioral windows could be detected. For each session, mice were recorded for 3 minutes of baseline in their home cage, then during the three social encounters. Mice were recorded in an open field for 5 minutes after each session. The order of the three social encounters were shuffled for each session. During each condition, the CD1 mouse received two segments of alternating blue (473 nm) and yellow (593.5 nm) light stimulation, for two minutes each.

For open-loop, CD1 mice received light stimulation for the entirety of the two-minute segment. For closed-loop, mice received stimulation for one second when reduced network score dropped below threshold.

For nonsynchronous stimulation, each CD1 mouse was randomly matched to a different mouse that had been used for closed-loop stimulation. Each non-synchronous mouse was then subjected to the identical order of conditions and yellow and blue light stimulation blocks as their individually matched closed-loop mouse. Light stimulation was delivered using the pattern implemented for the closed-loop partner mouse.

### Mediation Analysis

For the Baron and Kenny approach to establish that EN-AggINH expression mediated the behavioral effect of the neurostimulation, two results previously described in the methods were first used to establish that there was an effect from the neurostimulation on network expression and on behavior. The data used was next limited to the most relevant case, which is on the closed-loop stimulation. The data was limited to windows of LFP data during the closed-loop experiment where the laser was activated, either from the blue laser or the yellow laser to match the cases between the treatment and control as closely as possible. As only blue light should significantly manipulate neural activity, this is viewed as the treatment, and the yellow light is set as the control. “LFP preprocessing and signal artifact removal” was used to preprocess the data and remove data with significant artifacts. EN-AggINH expression was calculated by projecting the data into the learned model. The remaining data was then fit into two logistic regression models to predict behavior using the statsmodel package in python. The first model used only the stimulation to predict behavior (behavior~const+stimulation), and the second model used stimulation and network expression to predict behavior (behavior~const+network_expression+stimulation). These two models were compared by using a likelihood ratio test to evaluate whether the second model was significantly better.

For the causal mediation analysis, we again need to roughly balance treatment and control groups. We used the same data as described above in the classic mediation analysis. We define the treatment as blue versus yellow light stimulation, the mediator as EN-AggINH expression, and the outcome as aggressive versus non-aggressive behavior. Data was screened to remove windows with missing data.

### Multiple Brain Regions Fail to Independently Encode Attack Behavior Across Mice and Contexts

The initial analysis was focused on the medial prefrontal cortex (i.e., prelimbic and infralimbic cortex in mice), since this brain region had been implicated in social behaviors. Moreover, prior work had shown that optogenetic stimulation of the medial prefrontal cortex was sufficient to suppress attack behavior and increase non-aggressive social behaviors in CD1 strain mice. CD1 mice show periods of attack behavior, defined by biting, boxing (kicking/clawing), or tussling behavior, when a male C57BL6/J (C57) mouse is introduced into their home cage. On the other hand, they exhibit periods of non-attack social interactions such as sniffing, grooming, or resting (placing nose or forepaws against the subject mouse, but not moving) when exposed to a female intruder.

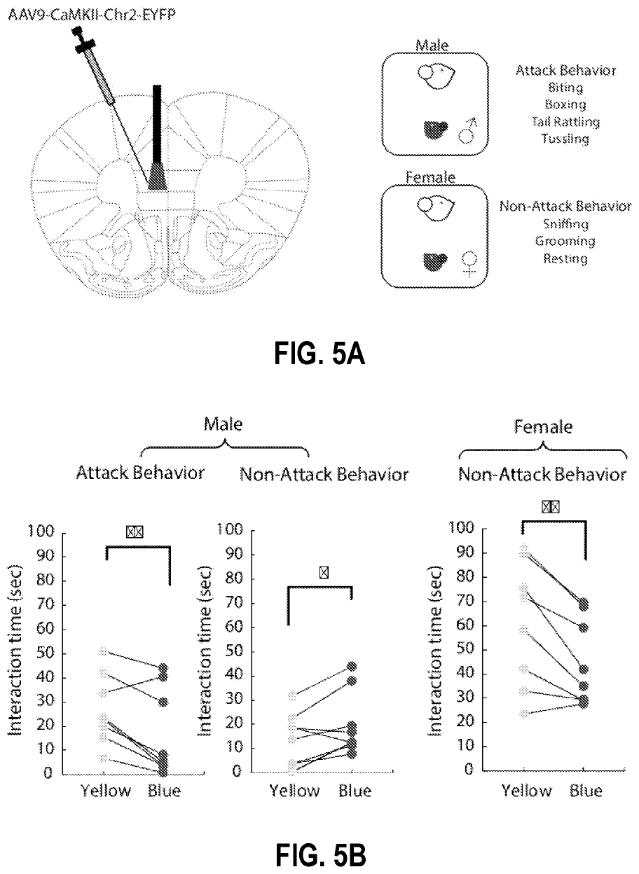

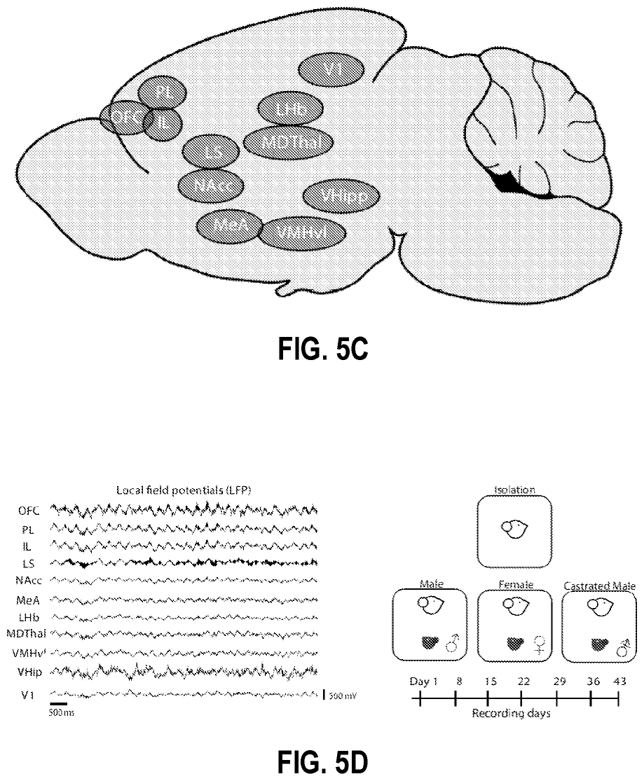

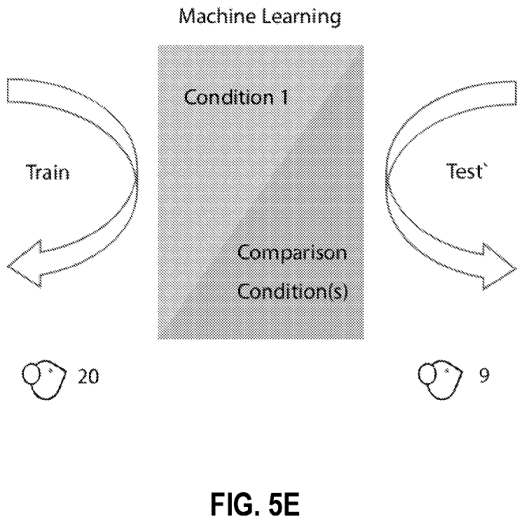

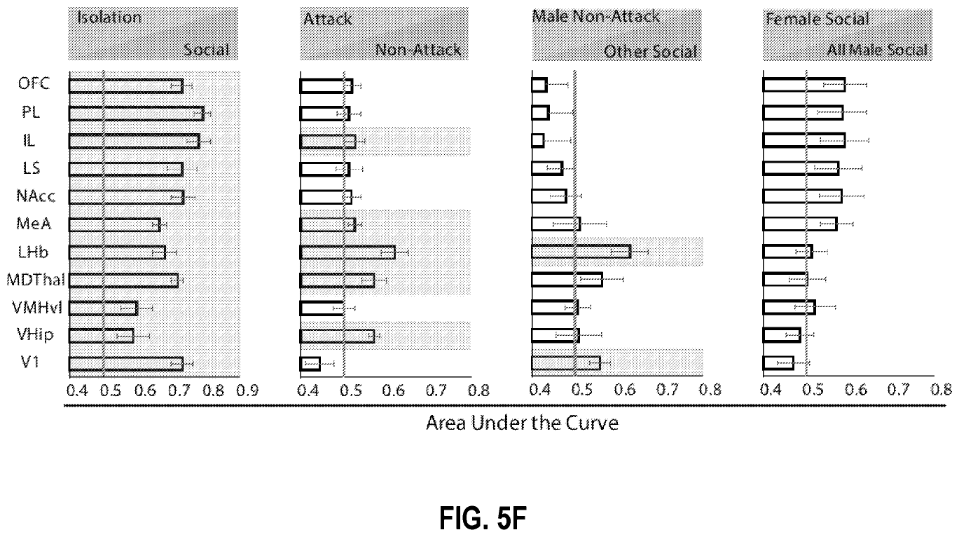

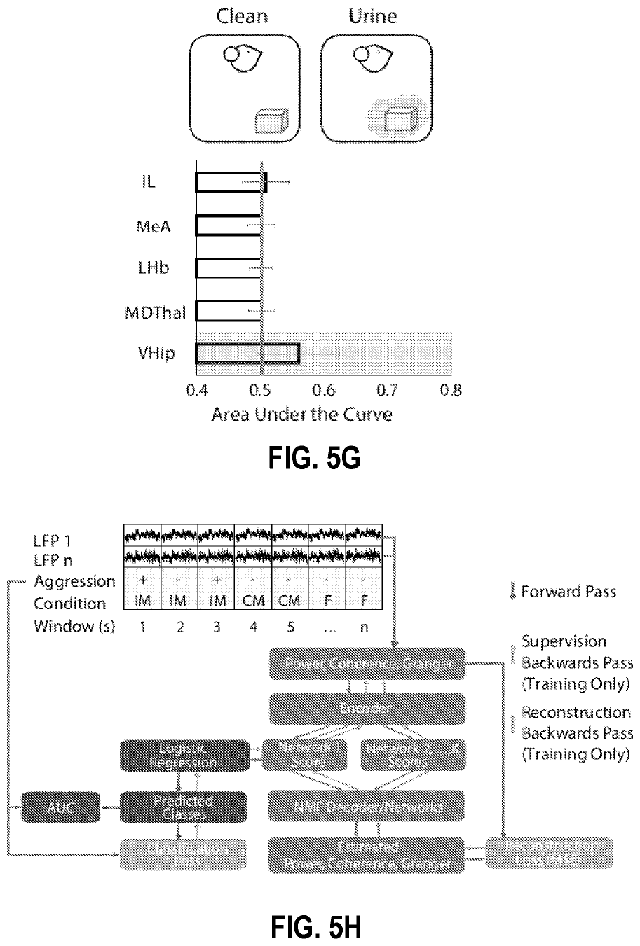

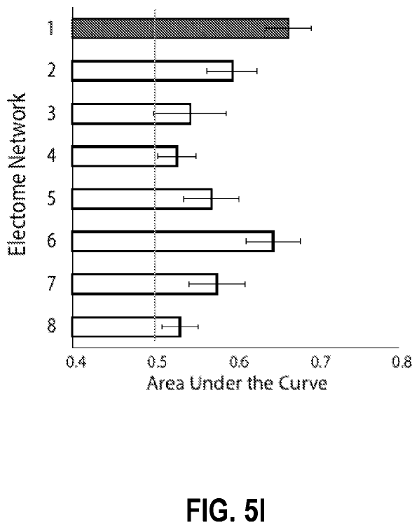

Optogenetic stimulation experiments were performed during these social encounters using a protocol modeled after prior work, where blue light stimulation is used to activate channelrhodopsin in the medial prefrontal cortex for the entirety of a social encounter (473 nm, 5 mW, 20 Hz, 3 ms pulse width, FIG. 5A-5B). Control experiments were performed using yellow light stimulation (593.5 nm, 5 mW, 20 Hz, 3 ms pulse width, FIG. 5A-5B), which does not activate channelrhodopsin. Consistent with the prior report, medial prefrontal cortex stimulation suppressed attack behavior towards C57 male mice and tended to increase non-attack social behavior [N=8; t7=3.43; P=0.0055, and t7=−2.35; P=0.051 using a two-tailed paired t-test, for attack and non-attack behavior, respectively, significance determined by a Benjamini-Hochberg false discovery rate (FDR) correction, FIG. 5B]. However, cortical stimulation decreased non-attack social behavior toward female intruders (t7=3.647, P=0.0041, using paired t-test, significance determined by FDR correction). Thus, medial prefrontal cortex stimulation was unable to selectively suppress attack behavior. Rather, stimulation suppressed multiple types of social behavior.

To probe for candidate brain regions that may exhibit such selectivity, CD1 mice were implanted with electrodes across multiple cortical and subcortical brain regions known to contribute to social behavior, including infralimbic cortex, orbitofrontal cortex, prelimbic cortex, lateral septum, nucleus accumbens, lateral habenula, mediodorsal thalamus, ventromedial hypothalamus, medial amygdala, primary visual cortex, and ventral hippocampus, as illustrated in FIG. 5C. Following surgical recovery, neural activity was recorded while the CD1 mice freely interacted with an intact male C57 mouse and a female C57 mouse for 300 seconds each. These encounters were repeated over six sessions, yielding a total of 1800 seconds of neural data and behavior for each exposure (FIG. 5D). A subset of these CD1 mice (N=9 of the 20 mice) were recorded as they interacted with a castrated male mouse intruder. Since CD1 mice do not generally exhibit attack behavior towards castrated males, this encounter provided neurophysiology data during additional non-attack social behaviors that were unpaired paired with female sensory cues. Neural activity was also acquired while the CD1 mice were isolated in their home cage.

It was first verified that each of the implanted brain regions encoded social behavior using discriminative cross spectral factor analysis non-negative matrix factorization (dCSFA-NMF, see FIG. 5H). dCSFA-NMF utilizes supervised machine learning to generate a statistical model that is both descriptive [integrates brain local field potential (LFP) activity features across time] and predictive (discovers networks that distinguish between types of external behavior). LFPs reflect the activity of populations of neurons, and these signals can be consistently sampled across mice. The electrical functional connectomes networks (Electome Networks) generated from dCSFA-NMF integrate LFP power (oscillatory amplitude across 1-56 Hz; a correlate of cellular and synaptic activity within brain regions), LFP synchrony (how two regions' LFP frequencies synchronize across time; a correlate of brain circuit function), and LFP Granger synchrony (Granger causality testing; a correlate of directional transfer of information across a brain circuit). Furthermore, dCSFA-NMF generates electome network activity scores (an indicator of the strength of each network) at a temporal resolution sufficient to capture brain states underlying the external behavior under observation (in this instance, a resolution of one second). The electome networks are designed to learn patterns that explain interpretable correlates of neural activity whose expression relate to measured behavior, facilitating an overall interpretable model. Any given brain region can belong to multiple electome networks and each electome network may incorporate any number of brain regions. dCSFA-NMF thus integrates spatially distinct brain regions and circuits into electome networks that encode behavior.

To explore whether there was a generalized activity pattern within individual regions that encoded social behavior, a series of dCSFA-NMF models were designed based on local field potential (LFP) oscillatory power in frequencies from 1-56 Hz. FIG. 5E shows Framework to test encoding of social states by individual brain regions. Each model was trained using observations pooled from twenty CD1 mice to separate periods where mice were socially isolated from periods where they were engaged in social behavior (e.g., attack behavior towards the intact males and non-attack social behavior towards male and female mice). The number of networks for each model was chosen by a validation procedure to balance how much brain activity is explained by the model, how much the model predicts behavior, and the parsimoniousness of the model (see methods). To facilitate easy interpretation of the learned networks, one network was encouraged to preferentially encode isolation from attack and non-attack social behavior (i.e., supervision). We tested the accuracy of the models in new CD1 mice recorded under all three conditions (N=9 mice).

High accuracy was observed in decoding periods of isolation from social behavior for each implanted brain area (p<0.05 using Wilcoxon rank-sum, significance determined by FDR correction for 44 comparisons, FIG. 5F). Next, a new series of models was designed to separate one of the three social behavioral states from the other three (e.g., attack behavior toward intact males vs. female, intact male, and castrated male non-attack interactions). These models were built using observations pooled from the same twenty CD1 mice, and again based on LFP power. The accuracy of the models was then tested in the same nine hold-out mice. Thus, the model's generalizability to decode each class of social behavior from the other three for each of the 11 implanted brain areas (i.e., 33 additional models) was trained and tested. Using this approach, it was found that five of the brain region-based statistical models decoded attack behavior versus non-attack social behavior: infralimbic cortex, lateral habenula, ventral hippocampus, medial amygdala, and medial dorsal thalamus. V1 successfully decoded the non-attack male interaction from the other social conditions as well (P<0.05 using one-tailed Wilcoxon rank-sum test, significance determined by FDR correction for 44 comparisons, see FIG. 5F). None of the other implanted brain regions showed this selectivity. Thus, five regions independently encoded attack behavior vs. non-attack social behavior in a manner that generalized across mice.

A broad goal was to identify a neural mechanism that could be used to suppress aggression while leaving other social behaviors intact. Thus, it was tested whether the putative attack codes discovered for the five regions generalized to another context associated with aggression. Specifically, urine from other male mice has been found to elicit aggressive and dominance behavior in CD1 males. As such, the most aggressive mice from the training and testing groups (N=8) were allowed to freely explore a clean inanimate object or an object covered in urine from another intact CD1 male mouse (seven sessions). It was then tested whether each of the five attack codes could distinguish periods where mice explored the clean objects from those where mice explored objects covered in urine. Though only ventral hippocampus model tended to decode behavior in this new context, none of the brain regions showed statistically significant encoding at the mesoscale-level (LFP) following correction for the five comparisons (AUC=0.56±0.06, P=0.04 for ventral hippocampus, using one-tailed Wilcoxon rank-sum test, significance determined by FDR correction for 5 comparisons, FIG. 5G).

### An Aggressive State is Encoded at the Network-Level

After failing to robustly decode attack behavior using LFPs independently from any of the 11 brain regions, it was established that a brain network integrated information across all the implanted brain regions to encode an aggressive state. This network-level encoding mechanism generalized to multiple new contexts associated with aggression. Critically, the network also encoded attack behavior with a predictive efficacy that exceeded independent ventral hippocampus activity.

For this analysis, a new dCFSA-NMF model was trained using data from all the implanted brain regions. The model utilized eight electome networks (see methods), one of which was trained to encode periods of attack-behavior (positive class) from social behavior in castrated male and female social context (negative class). Non-attack social behavior towards intact males were also included in the negative class to discourage the network from simply learning non-aggressive sensory cues specific to the intact male (i.e., supervision, electome network #1; see FIG. 5H). This network was then validated using the set of nine holdout CD1 mice from the single area coding test analysis. Again, none of these mice were used to train the electome networks.

It was found that electome network #1 successfully discriminated between attack behavior and non-attack social behavior in the test mice (N=9, Wilcoxon signed rank, p=0.0020, FIG. 5H-5I). Thus, the supervised network generalized to new animals.

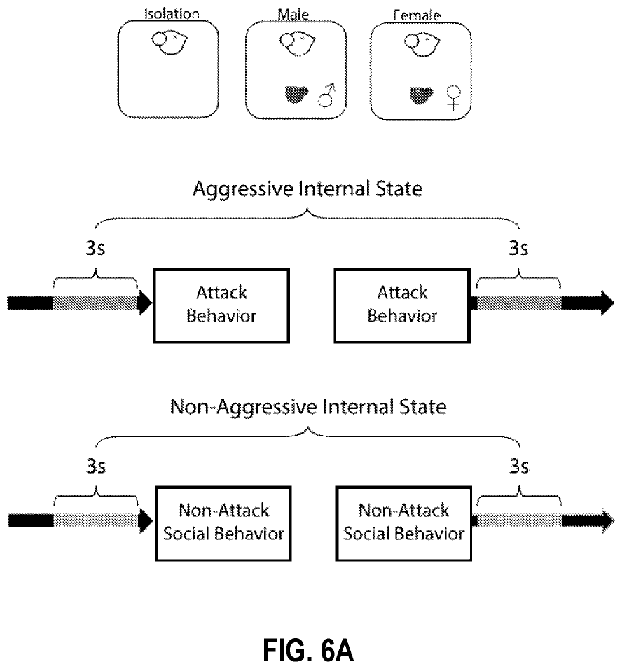

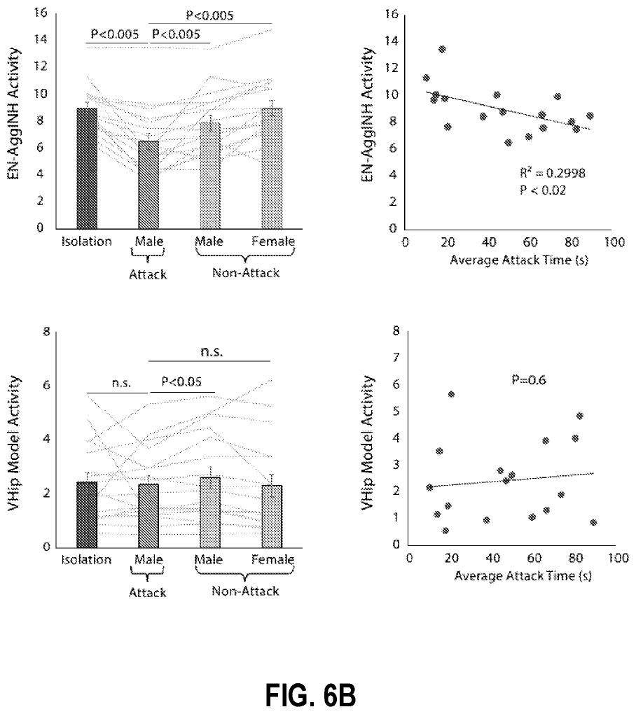

Attack behavior is indicative of an aggressive brain state. It was also reasoned that it was possible for a mouse to be in an aggressive internal state though it was not actively exhibiting attack behavior. Since such a context was likely to be present immediately prior to or following attack behavior, it was tested whether network activity pooled from the 6 seconds surrounding social behaviors encoded the distinct social conditions (FIG. 6A). Critically, these data windows were not used to train the network model since they did not contain attack or non-attack social behavior. Network activity was lower in the intervals surrounding attack behavior compared to non-attack social interactions with males or females (F3,67=28.6, P<0.0001 using Friedman's test, followed by P<0.05 using Wilcoxon signed-rank test, significance determined by FDR correction, FIG. 6B). Strikingly, network activity during periods of isolation also negatively correlated with the time mice spent exhibiting attack behavior towards other males (R=−0.58; P=0.016 using Spearman's rank correlation, FIG. 6B), encoding aggression on a mouse-by-mouse basis. Thus, electome network #1 (hereafter referred to as EN-Aggression Inhibition-AggINH) represented a network that putatively inhibited aggression when its activity was highest. In contrast to the models developed for each of the brain regions independently, EN-AggINH activity also encoded the exposure to male urine (N=8, Network Activity=9.1±1.0 and 8.3±1.1 for clean and urine covered objects, respectively; P=0.012 using one railed Wilcoxon signed-rank test; AUC=0.57±0.02, data not shown). Thus, EN-AggINH generalized to a second aggression context.

Next, the model was tested for ventral hippocampus since we observed a trend towards decoding attack behavior in the urine context. This model failed to encode the aggressive state. Specifically, activity from hippocampus was statistically indistinguishable between periods surrounding attack behavior and non-attack social interactions with females and activity during social isolation (F3,67=7.9, P=0.047 using Friedman's test; P=0.36 and 0.72, respectively, using Wilcoxon signed-rank test, significance determined by FDR correction, FIG. 6B). Moreover, there was no relationship between hippocampal activity during isolation and the innate aggressiveness of mice (P=0.60 using Spearman's rank, FIG. 6B). Thus, only network activity encoded the aggressive internal state.

### EN-Aggression Inhibition Maps to Interpretable Biology

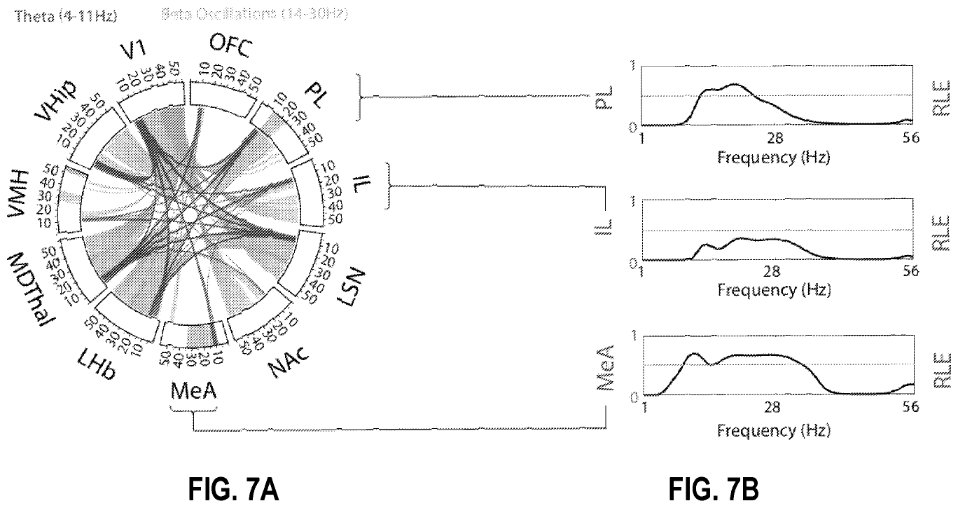

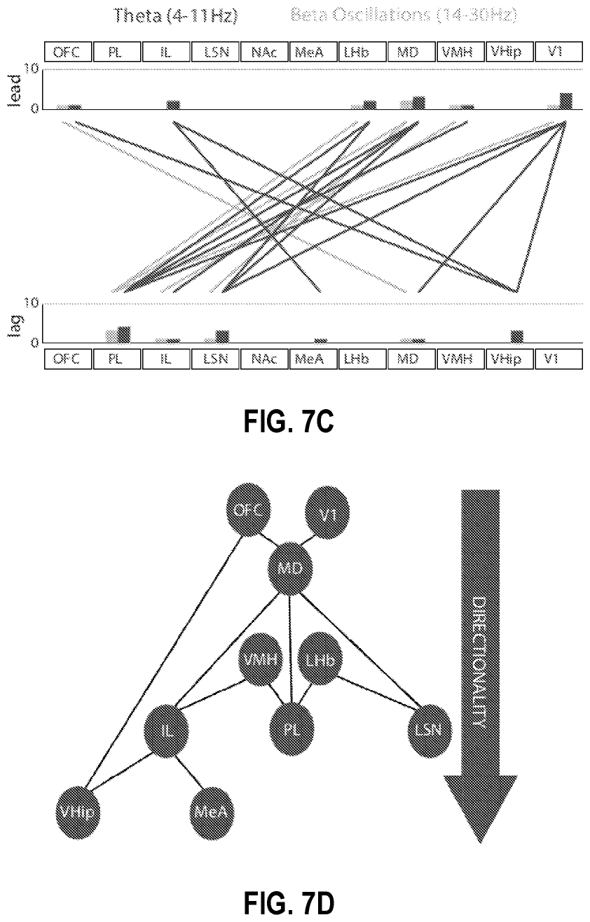

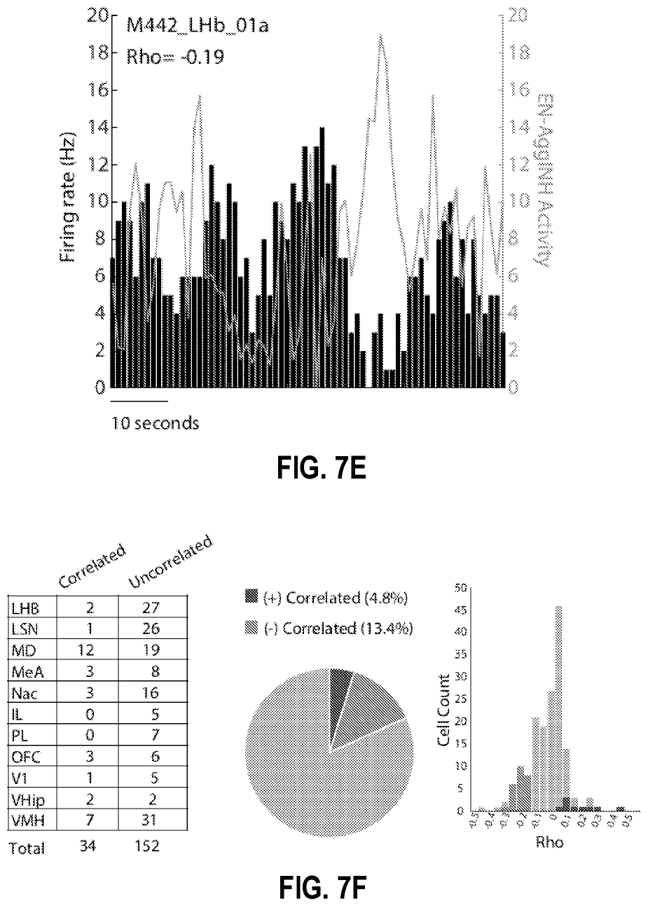

EN-AggINH was composed of prominent theta frequency activity (4-11 Hz) in medial amygdala and beta frequency activity (14-30 Hz) in medial amygdala and prelimbic cortex (FIG. 7A-7B). Prominent synchrony and directionality were also observed in the theta and beta frequency bands. Indeed, the network showed strong directionality that led from orbital frontal cortex and primary visual cortex, relayed through medial dorsal thalamus, and infralimbic cortex, and converged on medial amygdala and ventral hippocampus (FIG. 7C-7D).

It was verified that the activity of EN-AggINH truly reflects biological activity, by relating the electome network to neural firing, as in previous work. To achieve this, the correlation between cellular activity across the implanted brain regions and the activity of EN-AggINH was analyzed, as cell activity is an undisputed measure of biological function. A permutation test was used to rigorously test the findings (FIG. 7E). Specifically, cellular firing was shuffled within social behavioral conditions, maintaining the relationship between cell firing and behavior. This procedure was then repeated 1000 times to generate a null distribution for which only 5% of cells would be expected to exhibit firing coupled to network activity. It was found that ~18% of cells showed firing that was coupled to the activity of EN-AggINH, far more than could be explained by chance. Specifically, of the 186 cells recorded, nine (4.8%) showed firing activity that was positively correlated with EN-AggINH and 25 (13.4%) showed activity that was negatively correlated (FIG. 7F). Thus, it was confirmed that EN-AggINH activity reflects the dynamics of cellular activity across the brain.

### EN-Aggression Inhibition Generalizes to New Biological Contexts Related to Aggression

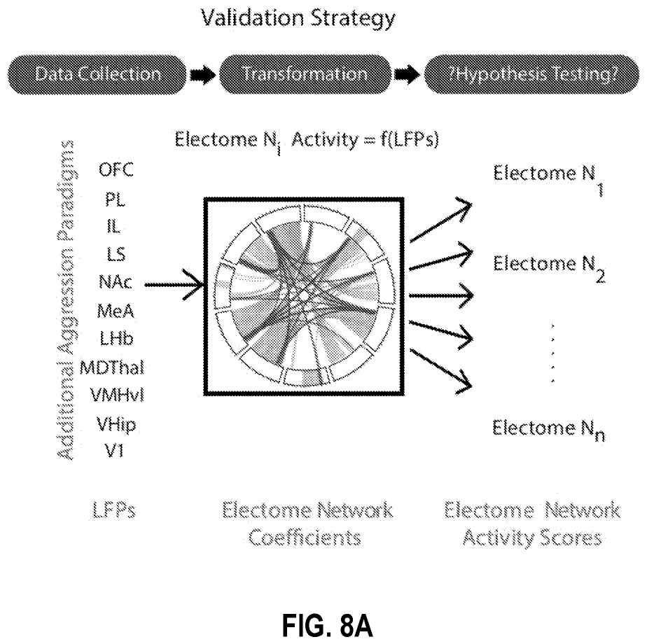

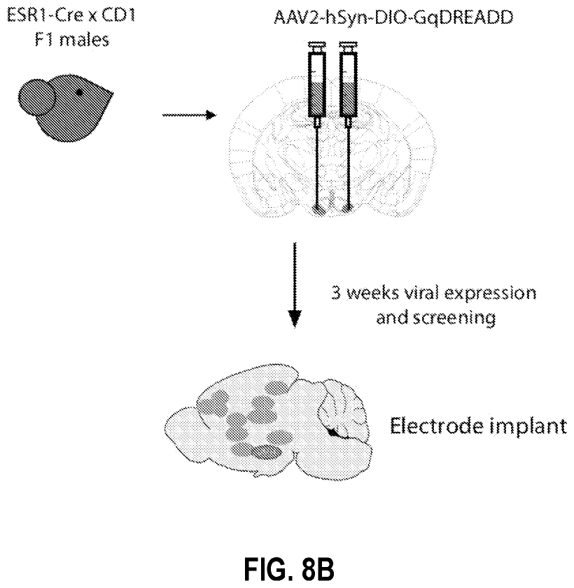

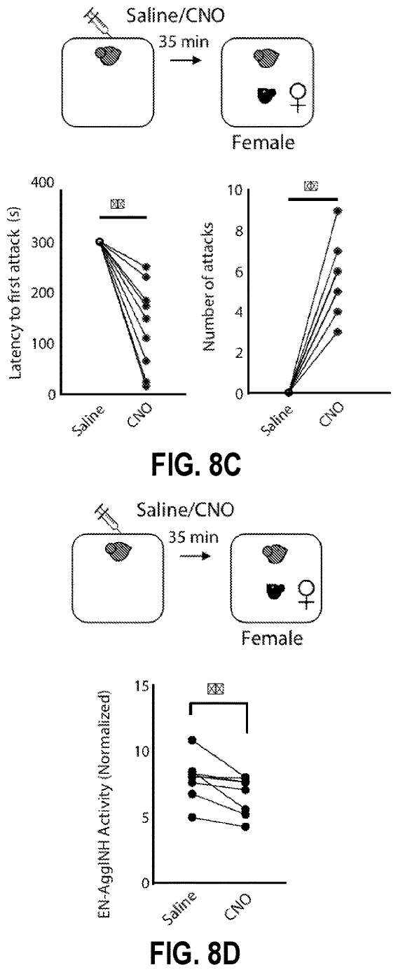

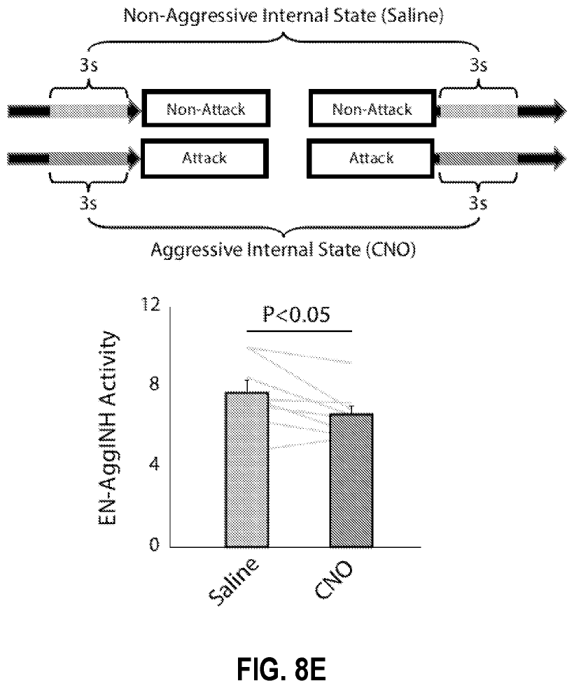

To further validate EN-AggINH, it was established that activity in this network was modulated by orthogonal biological conditions that have been shown to induce or suppress aggressive behavior in mice. In most cases, this analysis was performed in new animals, which is considered the gold standard of model validation in machine learning by experimental manipulation. LFP data recorded from these new sessions was transformed into the original network model (FIG. 8A).

A high-threshold validation experiment was first performed that involved testing whether the network generalized to new mice. These mice were on different genetic background and subjected to a new aggression context (FIG. 8B). Specifically, this approach also used a validated cellular manipulation that causally induces aggression under a behavioral condition that would otherwise not yield aggressive attack behavior (i.e., female social partners were used). An excitatory DREADD (AAV-hsyn-DIO-hM3Dq) was expressed in the ESR1+ cells of ventromedial hypothalamus, since it has been shown that direct excitation of these cells induces aggressive behavior towards female mice. Experiments were performed in the male F1 offspring of female CD1 strain mice crossed with ESR1-Cre male mice on a C57 strain background. Subsequently, the mice were implanted with recording electrodes to target the same brain regions as the initial experiment used to train the network model. Following recovery, behavioral and neural recording were performed when mice were exposed to a female mouse. The experimental mice were either treated with saline or CNO (Clozapine N-oxide, which activates the excitatory DREADD), in a pseudorandomized order, prior to the repeated testing sessions.

As anticipated, treatment with CNO induced attack behavior towards the female mice (N=8; P=0.0039 for both attack latency and attack number using one-tailed Wilcoxon sign rank; FIG. 8C). When neural activity was probed across the entire exposure to the female intruder, it was found that treatment with CNO also suppressed EN-AggINH activity (N=8, P=0.0039 using one-tailed Wilcoxon sign-rank; FIG. 8D). Thus, the network model generalized to a second aggression context induced by a cellular manipulation, and robust to different genetic backgrounds. Critically, network activity was also lower during the time intervals surrounding attack/non-attack social behavior for the CNO vs. saline treatment sessions (P=0.02 using one-tailed Wilcoxon sign-rank; FIG. 8E), again demonstrating the EN-AggINH encoded an aggressive state. EN-AggINH does not simply encode sensory cues associated with male intruders since the network responses observed in the CNO treated mice were induced by a female intruder.

### EN-Aggression Inhibition Mediates Attack Behavior

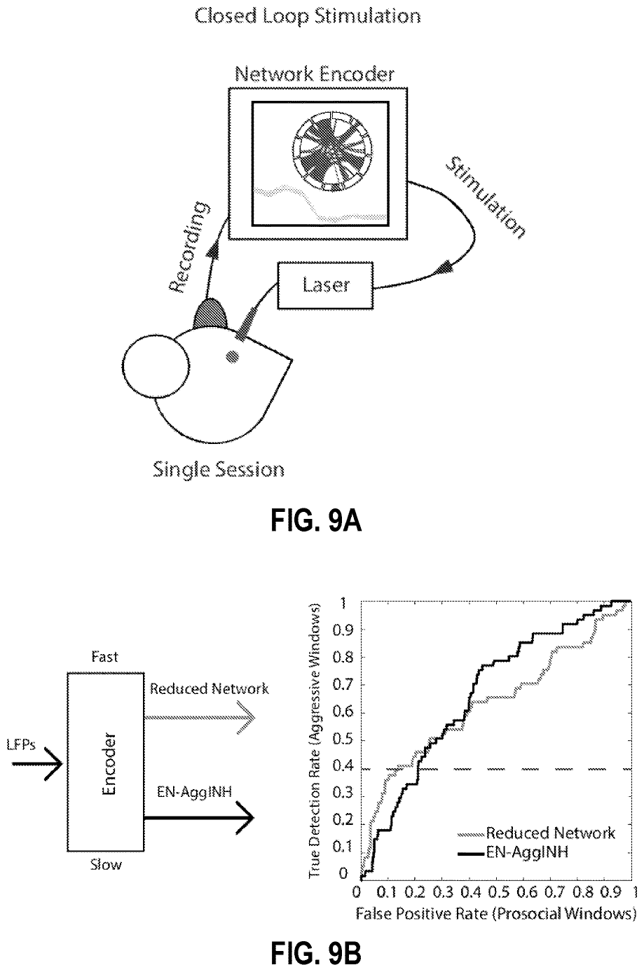

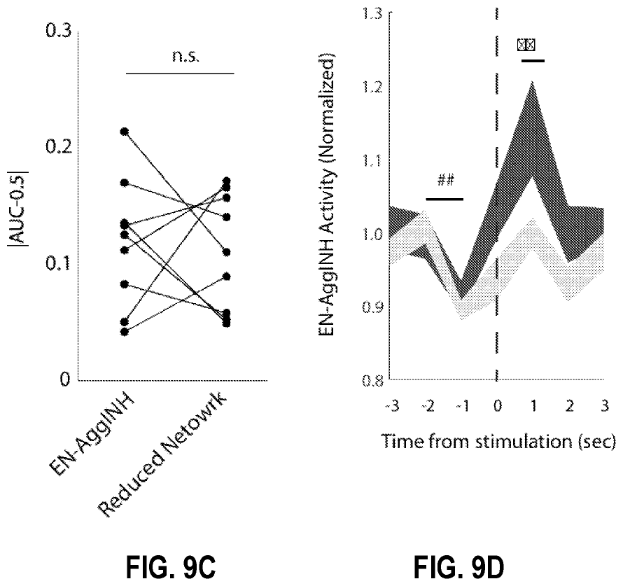

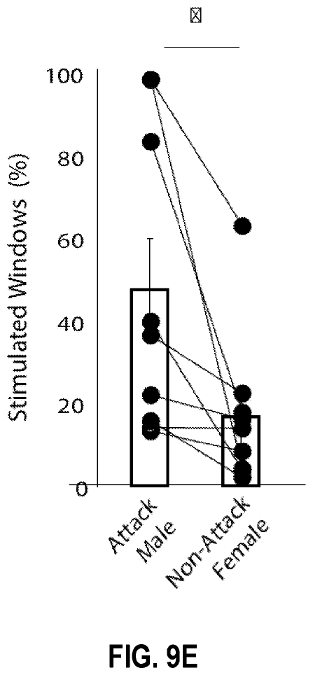

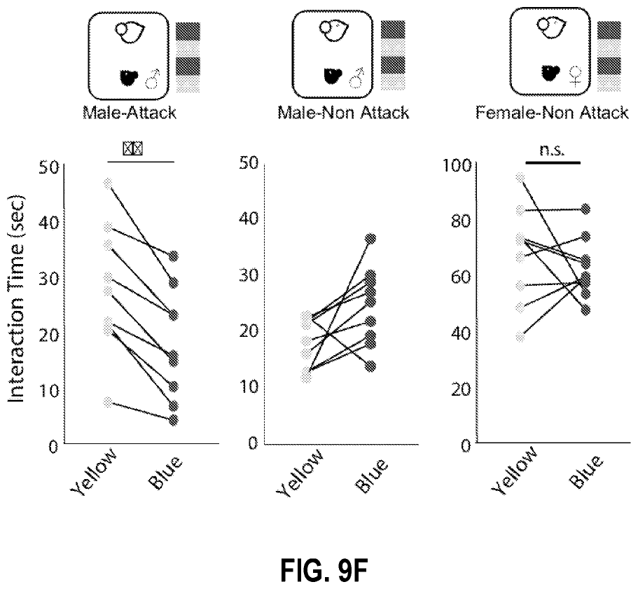

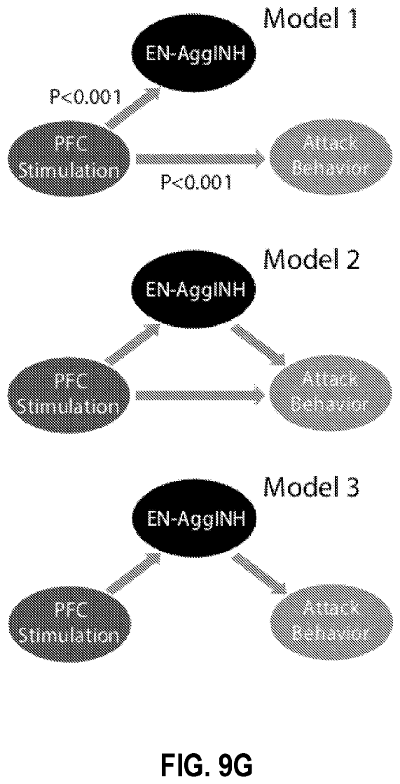

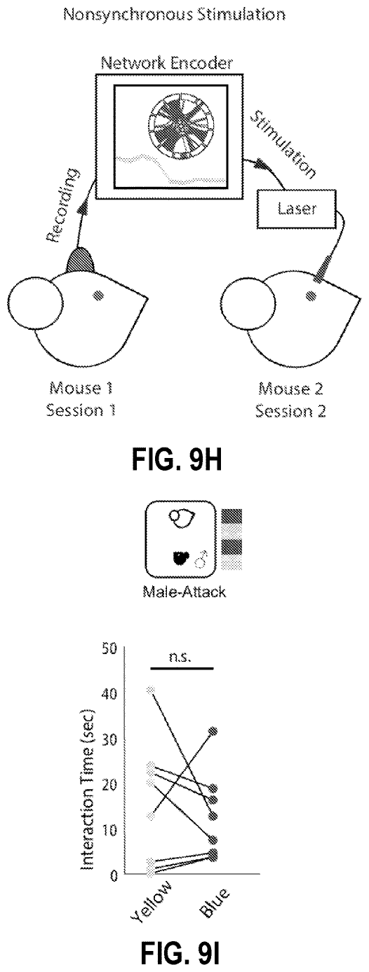

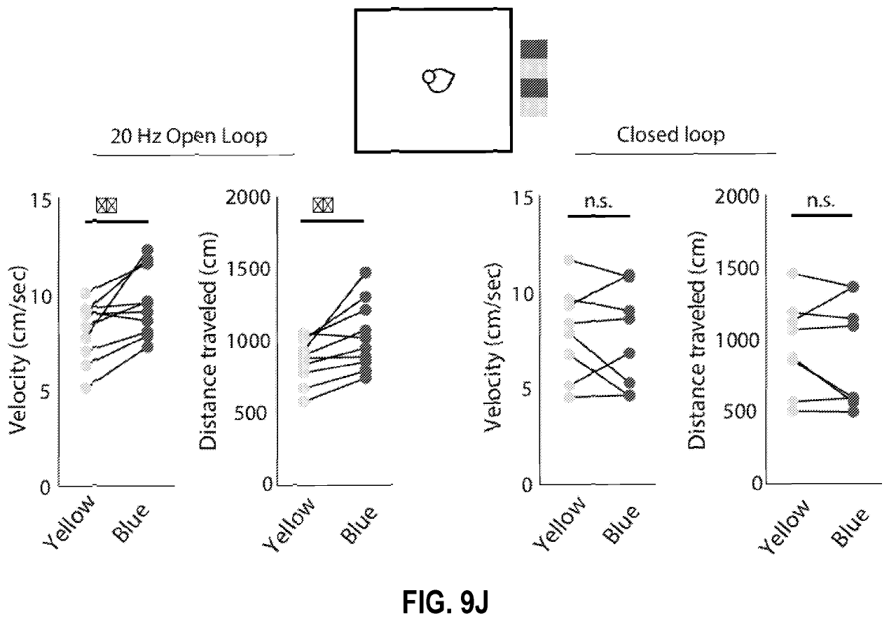

It was tested whether a cellular manipulation known to causally decrease aggression increased network activity. For these experiments, the medial prefrontal cortex was stimulated when EN-AggINH was naturally suppressed in the brain (signaling the aggressive state). First, a closed loop system was built that estimated the activity of EN-AggINH in real time (FIG. 9A). This approach employed a new network encoded solely based on power and coherence measures (i.e., a reduced network, FIG. 9B). While this new network lacked the interpretive power of dCSFA-NMF, it enabled prediction of aggression states in real time (FIG. 9C). In principle, when the activity of EN-AggINH fell below an established threshold (signaling the onset of aggressive behavior), the closed loop approach would deliver a one second light stimulation (5 mW, 20 Hz, 3 ms pulse width) to the prefrontal cortex. To verify that this real-time estimation system worked as designed, it was tested whether light stimulation was triggered by a decrease in EN-AggINH activity. Network activity was significantly lower one second prior to stimulation than it was two seconds prior to stimulation, demonstrating that the approach successfully identified when the EN-AggINH activity decreased below the threshold that signaled the onset of aggression (N=9; P<0.005 for within-subject comparison of EN-AggINH activity 1 vs 2 seconds prior to yellow light stimulation using one-tailed signed-rank test, FIG. 9D, note that activity was normalized for each mouse to the average network activity during isolation). Importantly, it was found that prefrontal cortex stimulation acutely increased EN-AggINH activity (N=9, P<0.01 for comparison of EN-AggINH activity one second after blue vs. yellow stimulation, using one-tailed signed-rank test, see FIG. 9D). Thus, it was confirmed that two cellular manipulations that causality regulated aggression also bidirectionally impacted AggINH activity.

Next, it was wondered whether increasing EN-AggINH activity via prefrontal cortex stimulation as the brain transitioned into a putative attack state would suppress aggressive behavior. It was also hypothesized that this approach would not alter non-attack social behavior during interactions with females. Specifically, since EN-AggINH activity was not naturally suppressed during female interactions it was anticipated that the closed loop protocol would deliver less stimulation to the brain (see FIG. 9E). Using this approach, it was found that closed loop stimulation of the prefrontal cortex significantly suppressed attack behavior (N=9 mice that were not used to train the initial model; t8=6.1, P=0.0003, FIG. 9F) and increased non-attack behavior ([N=9; 8=−2.3, P=0.049 comparing blue vs. yellow light stimulation using two-tailed paired t-test for attack behavior and non-attack behavior], significance determined by FDR correction, FIG. 9F) towards the C57 intact males. No differences in non-attack social behavior were observed during exposure to female mice (t8=0.74, P=0.48 using two-tailed paired t-test, significance determined by FDR correction, FIG. 9F).

Next, statistical methods were used to determine if the change in EN-AggINH activity was a mechanistic route for how closed loop stimulation regulated behavior. Mediation analysis was employed, which is a framework to determine whether the impact of a “treatment” (stimulation) on an outcome (aggressive behavior) is mediated by a change in an intermediate variable (EN-AggINH activity). If so, the intermediate variable is viewed, at least in part, as a mechanistic route (a mediator) for how the treatment impacts the outcome. First, the classic Baron and Kenny approach was used to determine whether EN-AggINH activity mediates the effect of neurostimulation on aggressive behavior. According to this statistical approach, there is a mediated effect of network activity on behavior if three conditions are met: 1) stimulation modulates network activity, 2) network activity correlates with behavior, and 3) modeling the behavior from network activity and stimulation together is better than modeling behavior from stimulation alone. A significant direct effect of stimulation on attack behavior (P<0.005, FIG. 9F) and network activity (P<0.0005, FIG. 9D) was previously identified. In order to match the conditions between the treatment and control cases as closely as possible, we chose to use windows during the closed-loop stimulation procedure where the laser was triggered, and then compared blue laser stimulation (treatment) to yellow laser stimulation (control). A statistical model of behavior using network activity and stimulation (see FIG. 9G, model 2) significantly outperformed the model using only stimulation (see FIG. 9G, model 1; nested logistic regression models, P<0.01, likelihood ratio test), satisfying the necessary conditions to show that EN-AggINH is a mediator.

After establishing that EN-AggINH activity mediated the impact of PFC stimulation on behavior, the significance of the average causal mediation effect (ACME) and the average direct effect (ADE) during the same stimulated closed-loop windows was evaluated using causal mediation analysis. ACME is the causal effect of stimulation on behavior due to the change in EN-AggINH activity (see. FIG. 9G, model 3), and ADE is the causal effect on behavior from prefrontal cortex stimulation not explained by the change in EN-AggINH activity. It was found that there was a significant ACME (P<0.01), but not a significant ADE (P=0.48). This analysis suggested that EN-AggINH activation is the primary mechanism whereby prefrontal cortex stimulation suppresses aggression.

An additional control experiment was performed to verify that the suppression of aggressive behavior observed in response to closed loop stimulation was due to the synchronization of the light stimulation and endogenous EN-AggINH activity, and not simply due to the dynamic pattern of stimulation delivered using this method. Here, the stimulation patterns from the closed loop experiments were used to drive stimulation in a new group of animals. Thus, for these sessions, prefrontal cortex stimulation occurred in a manner that mirrored the closed loop stimulation experiments, except that stimulation was not fixed to transitions into the aggressive internal state (i.e., open loop-nonsynchronous; FIG. 9H). Strikingly, this pattern of stimulation did not impact aggressive behavior (t7=0.64, P=0.54 using paired t-test; see FIG. 9I). Together with the mediation analysis, these results suggest that EN-AggINH activity plays a mechanistic role in mediating aggression.

Finally, since prior studies had also shown that prefrontal cortex stimulation induced hyperactivity, it was tested whether the suppression of aggressive behavior that resulted from prefrontal cortex stimulation was also associated with behavioral hyperactivity. While it was found that 20 Hz tonic stimulation of prefrontal cortex induced behavioral hyperactivity in the CD1 mice (N=10; T9=3.221 and P=0.0052 for Velocity; T9=3.213 and P=0.0053 for distance traveled, using one-tailed paired t-test; FIG. 9J), closed loop stimulation of prefrontal cortex had no impact on gross locomotor behavior (N=9; T8=0.584 and P=0.29 for velocity; T8=0.86 and P=0.21 for distance traveled, using one-tailed paired t-test; FIG. 9J). Thus, closed loop stimulation suppressed aggressive behavior, but it did not induce the off-target effects of impaired pro-social behavior and psychomotor activation that were observed with the classic 20 Hz stimulation paradigm.

### DISCUSSION

Behavior results from the integration of sensory information with the internal state of an animal. While many cellular substates that contribute to social behavioral outputs have been identified, less is known regarding the internal brain states that map sensory inputs to these behaviors. Here, it was set out to discover the internal state that regulates whether an animal will exhibit aggressive or pro-social behavior. It was reasoned that attack behaviors emerge from an aggressive internal brain state. Thus, machine learning was used to discover the mesoscale neural architecture of the brain when an animal exhibited attack vs. non attack social behaviors. Like other well defined internal brain states, such as sleep, it was found that the network distinguishing attack behavior incorporated state dependent patterns of neural activity across every brain region measured. For multiple regions, differences were observed in local oscillatory power, while others exhibited differences in oscillatory synchrony with a broader collection of regions. Each brain region showed selectivity in the frequencies of oscillations that contributed to the network. For example, prelimbic cortex showed strong activity in the beta frequency range, while medial amygdala showed strong activity in the theta frequency range. No brain region showed prominent activity contributions across all frequencies. It was also observed differences in the activity profile of a primary sensory region, V1, which may reflect a change in encoding, or differences in sensory input observed during attack behavior. Critically, the brain state identified during attack behavior was better captured by observing activity across all the brain regions as an integrated network, than observing independent activity within each brain region.

Though behavioral output has been classically utilized to infer the internal state of a brain, it was reasoned that an internal brain state was also likely present during intervals immediately surrounding behavioral output. Thus, it was tested whether the ‘attack’ network showed distinct activity profiles in the time intervals surrounding attack and non-attack social behaviors. Indeed, network activity was lower during an interval surrounding attack behavior. Strikingly, it was also found that network activity when animals were isolated in their home cage encoded their trait aggression. Thus, the network did not simply encode behavioral output since it was observed outside of attack behavior. Rather the network encoded an aggressive internal brain state.

Interesting, this aggressive brain state was encoded by decreased activity in the network. This observation does not indicate that overall brain activity is suppressed during aggressive states. Rather, the findings argue that the aggressive state is encoded by a network that decreases its activity relative to when mice are socially isolated or engaged in pro-social behavior. Indeed, the data suggested that several common regions/circuits were activated during aggressive and pro-social behavior. These common circuits need not be reflected in the network since the model was trained to differentiate attack vs. non-attack social behavior. Nevertheless, the discovery of a network that decreased its activity during aggression raises the intriguing hypothesis that this brain activity inhibits aggression during pro-social engagement. When activity in this inhibition network is suppressed, aggressive behavior emerges.

This interpretation is supported by the validation experiments where ESR1+-Cre neurons were directly activated in ventromedial hypothalamus. The findings showed that direct activation of these cells induced the aggressive brain state (suppressed EN-AggINH activity). When mice treated with CNO were exposed to a stimulus that would generally produce non-attack social behavior (i.e., a female mouse), attack behavior emerged. Thus, the presence of the aggressive brain state changed the mapping between sensory input and behavior output. Similarly, direct stimulation of medial prefrontal cortex biased mice towards exhibiting non-attack social behavior when they were exposed to a stimulus that would generally induce attack behavior (i.e., a male intruder). The findings showed that medial prefrontal cortex stimulation decreased the aggressive internal state (increased EN-AggINH network activity). Critically, the findings using mediation analysis argue that the brain state represented by EN-AggINH is mechanistically involved in the translation of medial prefrontal cortex stimulation to a suppression of attack behavior.

Here, internal brain states were framed as a collection of functions that transform sensory input into behavior. Indeed, it was found that when EN-AggINH activity is suppressed, the brain transforms both male and female social sensory cues into attack behavior. It is also widely appreciated that sensory input can also cause the brain to transition from one internal state to another. For example, a loud sound can cause an animal to transition from sleeping to a hyper aroused internal state. Along this line, it was found that exposure to male mice could promote an aggressive internal state in CD1 mice, even prior to attack behavior, while exposure to a female mouse did not (under normal conditions). In this framework, many modulatory strategies that regulate attack behavior could mediate their effect by driving the brain out of the brain state represented by low EN-AggINH activity. For example, delivering a bright visual cue or a strong sensory cue (i.e., air puff) timed to decreases in EN-AggINH activity may be used suppress attack behavior, since many circuits and sensory inputs likely converge onto the internal state represented by EN-AggINH.

The closed loop stimulation approach was developed using a neural-network based approximation technique that was substantially constrained relative to dCSFA-NMF. Specifically, the reduced encoder was developed solely using power and coherence features. This approach was needed because the processing time to calculate granger directionality was prohibitive for real time implementations. As such, the reduced encoder sacrifices some predictive efficacy for speed. Nevertheless, it was found that the reduced encoder was sufficient to identify the precise time windows where the brain transitioned into aggression, as marked by a decrease in EN-AggINH activity. In some examples, convolutional neural networks may be used to bypass the feature extraction step to further improve the precision of the real time stimulation approach. These neural network encoders may be altered to predict both aggressive and pro-social states, such as the generalized social appetitive network. By using both networks concurrently to actuate a closed loop system, aggressive behavior could be further suppressed relative to pro-social behavior. Indeed, the findings also pointed to a network that exhibits increased activity during aggressive behavior (Electome Network 6, see FIG. 6B). This network showed strong encoding in the nine hold out test mice (AUC-0.65±0.03, P<5×10-5 using Wilcoxon rank sum), and activity in the network also generalized to the experiment in which ventromedial hypothalamus activity was causally manipulated. Though the network failed to encode the urine paradigm, it is possible that it contains activity that synergizes with EN-AggINH to encode aggressive social states more optimally. In an example, imitation encoders for both Electome Network 6 and EN-AggINH may be integrated to further optimize closed loop approaches to selectively suppress aggression.

Multiple neuropsychiatric disorders including mood disorders, psychotic disorders, neurodevelopmental disorders, and neurodegenerative disorders are associated with deficits in regulating social behavior, including aggression. While multiple pharmacological approaches have been instituted to suppress aggressive behavior towards self and others, many of these strategies act by sedating the individual and can disrupt aspects of pro-social function. The discovery of a brain network that encoded an aggressive state raises the potential for novel approaches to suppress aggressive behavior that spare pro-social behavior. Indeed, compared to a standard open loop stimulation protocol (20 Hz stimulation) which suppressed both attack and non-attack pro-social behavior, the closed loop stimulation approach spared non-attack social behavior towards males or females. Intriguingly, open loop stimulation pattern (20 Hz stimulation) also induced behavioral hyperactivity in experimental mice, while the closed loop stimulation protocol did not. Thus, the findings also show that closed loop stimulation may limit off target behavioral effects that are induced by classic stimulation approaches.

Overall, the findings establish a generalized network-level mechanism whereby the brain suppresses aggression via active inhibition. Moreover, they highlight the potential for state-specific neuromodulation to regulate internal states.

### V. CONCLUSION

The particular arrangements shown in the Figures should not be viewed as limiting. It should be understood that other embodiments may include more or less of each element shown in a given Figure. Further, some of the illustrated elements may be combined or omitted. Yet further, an illustrative embodiment may include elements that are not illustrated in the Figures.

A step or block that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical functions or actions in the method or technique. The program code and/or related data can be stored on any type of computer readable medium such as a storage device including a disk, hard drive, or other storage medium.

The computer readable medium can also include non-transitory computer readable media such as computer-readable media that store data for short periods of time like register memory, processor cache, and random access memory (RAM). The computer readable media can also include non-transitory computer readable media that store program code and/or data for longer periods of time. Thus, the computer readable media may include secondary or persistent long term storage, like read only memory (ROM), optical or magnetic disks, compact-disc read only memory (CD-ROM), for example. The computer readable media can also be any other volatile or non-volatile storage systems. A computer readable medium can be considered a computer readable storage medium, for example, or a tangible storage device.

While various examples and embodiments have been disclosed, other examples and embodiments will be apparent to those skilled in the art. The various disclosed examples and embodiments are for purposes of illustration and are not intended to be limiting, with the true scope being indicated by the following claims.

What is claimed is:

1. A system for predicting and treating a neurological behavior in a subject comprising:
a plurality of electrodes configured to receive electrical activity signals from a network of two or more regions of the subject's brain; and
a controller in communication with the plurality of electrodes, the controller having at least one processor in communication with a memory, the memory including instructions executable by the processor to:
receive and record the electrical activity signals from the plurality of electrodes;
generate a prediction of a time of onset of the neurological behavior from the electrical activity signals; and
send a stimulation signal to the brain prior to the predicted time of onset of the neurological behavior,
wherein the stimulation signal to the brain suppresses the neurological behavior.

2. The system of claim 1, the memory including further instructions to:
provide the electrical activity signals to a trained machine learning model, wherein the prediction of the time of onset is generated from the trained machine learning model.

3. The system of claim 1, wherein the plurality of electrodes are microwire electrodes.

4. The system of claim 1, wherein the two or more regions comprises 8 to 11 regions of the subject's brain.

5. The system of claim 1, wherein the two or more brain regions are selected from the group consisting of infralimbic cortex, orbitofrontal cortex, prelimbic cortex, lateral septum, nucleus accumbens, lateral habenula, mediodorsal thalamus, ventromedial hypothalamus, medial amygdala, primary visual cortex, ventral hippocampus, and combinations thereof.

6. The system of claim 1, wherein the neurological behavior is an aggressive state.

7. The system of claim 1, wherein the neurological behavior is an aggressive attack.

8. The system of claim 1, wherein the plurality of electrodes are implanted in the subject's brain.

9. The system of claim 1, wherein the time of onset of the neurological behavior is on a second timescale.

10. The system of claim 1, further comprising a stimulator configured to generate the stimulation signal.

11. The system of claim 10, wherein the stimulation signal is light stimulation.

12. The system of claim 10, wherein the stimulation signal is directed to the prefrontal cortex of the subject's brain.

13. The system of claim 12, wherein the stimulation signal comprises closed loop optogenetic stimulation.

14. A method for predicting and treating a neurological behavior in a subject comprising:
providing a plurality of electrodes configured to receive electrical activity signals from a network of two or more regions of the subject's brain;
receiving and recording the electrical activity signals from the plurality of electrodes, via a controller in communication with the plurality of electrodes;
generating a prediction of a time of onset of the neurological behavior from the electrical activity signals; and
sending a stimulation signal to the brain prior to the predicted time of onset of the neurological behavior,
wherein the stimulation signal to the brain suppresses the neurological behavior.

15. The method of claim 14, further comprising:
providing the electrical activity signals to a trained machine learning model, wherein the prediction of the time of onset is generated from the trained machine learning model.

16. The method of claim 14, wherein the two or more regions comprises 8 to 11 regions of the subject's brain.

17. The method of claim 14, wherein the two or more brain regions are selected from the group consisting of infralimbic cortex, orbitofrontal cortex, prelimbic cortex, lateral septum. nucleus accumbens, lateral habenula, mediodorsal thalamus, ventromedial hypothalamus, medial amygdala, primary visual cortex, and ventral hippocampus.

18. The method of claim 14, wherein the neurological behavior is an aggressive state.

19. The method of claim 14, wherein the neurological behavior is an aggressive attack.

20. The method of claim 14, wherein the plurality of electrodes are implanted in the subject's brain.

21. The method of claim 14, wherein the time of onset of the neurological behavior is on a second timescale.

22. The method of claim 14, wherein the stimulation signal is directed to the prefrontal cortex of the subject's brain.

23. The method of claim 22, wherein the stimulation signal comprises closed loop optogenetic stimulation.

24. The method of claim 14, wherein the stimulation signal prevents the onset of the neurological behavior.
